(19) United States
(12) Patent Application Publication
(10) Pub. No.: US 2026/0355317 A1
(43) Pub. Date: Nov. 12, 2026

(54) CHIMERIC ANTIGEN RECEPTOR SYSTEMS, METHODS OF PREPARATION, AND USES THEREOF

(71) Applicants: WESTLAKE GENETECH. LTD., Hangzhou, Zhejiang (CN); WESTLAKE UNIVERSITY, Hangzhou, Zhejiang (CN)

(72) Inventors: Zhike LU, Hangzhou, Zhejiang (CN); Lijia MA, Hangzhou, Zhejiang (CN); Ke NI, Hangzhou, Zhejiang (CN)

(21) Appl. No.: 19/100,147

(22) PCT Filed: Aug. 2, 2023
(86) PCT No.: PCT/CN2023/110768

§ 371 Date: Jan. 31, 2025

(30) Foreign Application Priority Data
Aug. 3, 2022 (WO) PCT/CN2022/109857

(51) Int. Cl. A61K 40/31; A61K 35/17; A61K 40/10; A61K 40/41; A61P 35/00; C07K 16/28; C12N 5/0783; C12N 9/64

(52) U.S. Cl. CPC A61K 40/31; A61K 35/17; A61K 40/10; A61K 40/41; A61P 35/00; C07K 16/28; C12N 5/0636; C12N 9/6472; A61K 2239/13; A61K 2239/21; A61K 2239/22; C07K 2317/622; C12N 2510/00; C12Y 304/2207

(57) ABSTRACT

The present disclosure provides a Smart CAR system, where an exogenous antigen is labeled on tumor cells, the tumor cells labeled with the exogenous antigen can be recognized by engineered immune cells that express a chimeric receptor comprising a binding domain that binds the exogenous antigen, and the tumor can be eliminated at least in part. The present disclosure also provides methods of preparation, methods of treatment, and uses related to the Smart CAR system.

### RELATED APPLICATION

This application claims priority to International Application No. PCT/CN2022/109857, filed on Aug. 3, 2022, the content of which is incorporated by reference in its entirety.

### SEQUENCE LISTING

The instant application contains a Sequence Listing, which has been submitted electronically in an XML file entitled “Seq.xml” having a size of 31 KB and created on Aug. 1, 2023. The information contained in the Sequence Listing is hereby incorporated by reference in its entirety.

### FIELD

The present disclosure relates to treatment of a tumor wherein tumor cells are labeled with an exogenous antigen and are treated by engineered immune cells that express a chimeric antigen receptor (CAR) comprising a binding domain that recognizes the exogenous antigen.

### BACKGROUND

Although chimeric antigen receptor T (CAR-T) cell has achieved significant success in clinical trials for B-cell malignancies, its applicability for solid tumors is limited. Various factors contribute to the hurdle of making an efficacious CAR-T cell for solid tumors, and a lack of tumor-specific antigens is one of the main reasons (1, 2). Identification of antigens that have high, homogeneous expressions in solid tumors but not in healthy tissues has proven difficult (2). Current CAR typically comprises an extracellular antigen-binding domain, which is commonly a single-chain variable fragment (scFv). Screening of scFvs with appropriate affinity against a tumor antigen requires a tremendous effort. In addition, acquired resistance caused by heterogeneity and antigen loss in tumors renders universal applications of CAR-T cell therapy difficult (3, 4).

To overcome one or more of these problems, the present disclosure provides a Smart CAR system, wherein an exogenous antigen is labeled on the surface of tumor cells, the tumor cells labeled with the exogenous antigen can be recognized by engineered immune cells that express a chimeric receptor comprising a binding domain that binds the exogenous antigen, and subsequently the tumor can be treated, e.g., at least partially eliminated.

### SUMMARY

In one aspect, the present disclosure provides a method of treating a tumor in a subject in need thereof, comprising:
(a) providing a sortase and an exogenous antigen to the tumor, wherein the exogenous antigen comprises an exogenous antigen ligand and a sortag motif, and wherein cells in the tumor are sortagged by the exogenous antigen, and
(b) administering to the subject genetically engineered immune cells that express a chimeric antigen receptor (CAR) comprising an antigen binding domain that is capable of binding the exogenous antigen ligand.

In some embodiment, the CAR comprising an antigen binding domain that is capable of binding the exogenous antigen ligand further comprises a binding domain that is capable of binding an endogenous antigen in the tumor.

In some embodiment, the method of treating a tumor further comprises administering to the subject genetically engineered immune cells that express a second CAR, wherein the second CAR comprises a binding domain that recognizes an endogenous antigen or exogenous antigen in the tumor.

In one aspect, the present disclosure provides a method of labeling a tumor cell with an exogenous antigen, comprising providing a sortase and an exogenous antigen to the tumor cell, wherein the exogenous antigen comprises an exogenous antigen ligand and a sortag motif, and wherein the tumor cells is sortagged by the exogenous antigen.

In one aspect, the present disclosure provides a tumor cell sortagged with an exogenous antigen, e.g., by a method of labeling a tumor cell described herein.

In one aspect, the present disclosure provides an exogenous antigen comprising a sortag motif and an exogenous antigen ligand.

In one aspect, the present disclosure provides a nucleic acid encoding a CAR, wherein the CAR comprises an antigen binding domain, a transmembrane domain, one or more costimulatory signaling regions, and a cytoplasmic domain comprising an immunoreceptor tyrosine-based activation motif (ITAM), wherein the antigen binding domain is capable of binding an exogenous antigen described herein. In some embodiment, the CAR further comprises a binding domain that is capable of binding an endogenous or exogenous antigen in the tumor.

In one aspect, the present disclosure provides a vector comprising a nucleic acid described herein.

In one aspect, the present disclosure provides an engineered immune cell comprising a nucleic acid or a vector described herein.

In one aspect, the present disclosure provides a pharmaceutical composition comprising a population of engineered immune cells described herein.

In one aspect, the present disclosure provides a method of modifying an immune cell comprising delivering to the immune cell a vector or a nucleic acid described herein.

In one aspect, the present disclosure provides a kit comprising a sortase or a nucleic acid or vector encoding the same, and an exogenous antigen. In some embodiments, the kit further comprises a nucleic acid or a vector encoding a CAR described herein.

In one aspect, the present disclosure provides genetically engineered immune cells for treating a tumor, wherein cells in the tumor are sortagged by an exogenous antigen mediated by a sortase, wherein the exogenous antigen comprises an exogenous antigen ligand and a sortag motif, and wherein the genetically engineered immune cells express a CAR comprising an antigen binding domain that is capable of binding the exogenous antigen ligand. In some embodiment, the CAR further comprises a binding domain that is capable of binding an endogenous or exogenous antigen in the tumor.

In one aspect, the present disclosure provides a use of genetically engineered immune cells for the manufacture of a medicament for treating a tumor, wherein cells in the tumor are sortagged by an exogenous antigen mediated by a sortase, wherein the exogenous antigen comprises an exogenous antigen ligand and a sortag motif, and wherein the genetically engineered immune cells express a CAR comprising an antigen binding domain that is capable of binding the exogenous antigen ligand. In some embodiment, the CAR further comprises a binding domain that is capable of binding an endogenous or exogenous antigen in the tumor.

### BRIEF DESCRIPTION OF THE DRAWINGS

FIG. 1 shows a Smart CAR-T system (right) that comprises a mixture of a sortase enzyme and an exogeneous antigen, as well as Smart CAR-T cells, in comparison with a classical CAR-T system (left). FIG. 1A illustrates a Smart CAR-T system (right) that comprises Smart CAR-T cells having a TNFRSF9 cytoplasmic domain, in comparison to a classical CAR-T system (left).

FIG. 2 illustrates a vector design for an monomeric streptavidin (mSA)-CAR.

FIG. 3A shows transduction efficiency and mSA-CAR expression on the surface of T cells after 2 days, 8 days, and 11 days of transduction. The transduction efficiency was represented by the percentage of tagBFP+ cells. The mSA-CAR expression on the surface of T cells was detected by FITC-AhX-GGGGK(Biotin)-NH2 (SEQ ID NO: 9) through the binding of mSA and biotin.

FIG. 3B and FIG. 3C show the proliferation (FIG. 3B) and viability (FIG. 3C) of mSA-CAR-T cells 0 day (DO), 2 days (D2), 4 days (D4), 8 days (D8), and 11 days (D11) after lentivirus transduction. mSA-CAR-T: primary T cells with mSA-CAR expression; UN-T: primary T cells without mSA-CAR expression.

FIG. 4A shows quantification of Biotin-AALPETG*G (SEQ ID NO: 14) sortagged on various solid tumor cell lines. SORT: Tumor cells were sortagged with Biotin-AALPETG*G (SEQ ID NO: 14); Blank: Tumor cells without sortagging. The sortagging efficiency was indicated by the intensity of phycoerythrin (PE)-streptavidin.

FIG. 4B shows quantification of Biotin-AALPETG*G (SEQ ID NO: 14) on (left) Hela and (right) MDA-MB-231 cells at time points after an intra-tumor injection in vivo. SORT: tumor cells were sortagged with Biotin-AALPETG*G (SEQ ID NO: 14); Blank: tumor cells without sortagging. The sortagging efficiency was indicated by PE-streptavidin.

FIG. 5A shows expression of CD25 and CD69 on the surface of T cells while co-cultured with tumor cells. mSA-CAR-T: primary T cells expressing mSA-CAR; UN-T: primary T cell without mSA-CAR expression; SORT: tumor cells sortagged with Biotin-AALPETG*G (SEQ ID NO: 14); Blank: tumor cells without sortagging.

FIG. 5B shows cytokine release of T cells against tumor cells. mSA-CAR-T: primary T cells expressing mSA-CAR; UN-T: primary T cell without mSA-CAR expression; the terms that end with “Blank” in x-axis labels mean tumor cells without sortagging; the terms that end with “Sort” in the x-axis labels mean tumor cells sortagged with Biotin-AALPETG*G (SEQ ID NO: 14).

FIG. 5C shows cytotoxicity of mSA-CAR-T cells against Hela, MDA-MB-231, or HepG2 solid tumor cell lines with stable luciferase expression. Blank: tumor cells without sortagging; SORT: tumor cells sortagged with Biotin-AALPETG*G (SEQ ID NO: 14); -luc: with stable expression of luciferase.

FIG. 6 shows tumor size curves of mice receiving mSA-CAR-T cells as well as sortag intratumor injections. The vertical arrows indicated the timepoints of giving one dose injection of the respective sortag mixtures to the mice, in a total of 6 doses. mSA-CAR T+Biotin-motif: mouse received mSA-CAR-T cells and intratumor injection of sortag; UN-T+Biotin-motif: mouse received primary T cells and intratumor injection of sortag; Blank: mouse did not receive primary T cells and without receiving intratumor injection.

FIG. 7 shows a Smart CAR vector design for a PNEscFv-CAR, which recognizes PNE via an anti-PNE scFv. The Smart CAR modular design comprises PNE scFv, a hinge domain, a transmembrane domain, a co-stimulatory domain, and CD247.

FIG. 8A shows transduction efficiency and PNEscFv-CAR expression on the surface of T cells on different days post transduction. The transduction efficiency was indicated by tagBFP. The PNEscFv-CAR expression on the surface of T cells was indicated as PE-streptavidin.

FIG. 8B shows identification of Biotin-PNE sortagged on 293T cells. SORT: tumor cells sortagged; Blank: tumor cells without sortagging. The sortagging efficiency was indicated by anti-Biotin-PE.

FIG. 8C shows expression of CD25 and CD69 on the surface of T cells when co-cultured with tumor cells. PNEscFv-CAR-T: primary T cells expressing PNEscFv-CAR; UN-T: primary T cell without PNEscFv-CAR expression; SORT: tumor cells sortagged with Biotin-PNE; Blank: tumor cells without sortagging.

FIG. 9A and FIG. 9B show tumor size curves for mice receiving PNEscFv-CAR-T cells as well as sortag intratumor injections. The vertical arrows indicated the timepoints of giving one dose injection of the respective sortag mixtures to the mice. PNE-CAR-T+PNE-motif: mouse received PNEscFv-CAR-T cells and intratumor injection of sortag; UN-T+PNE-motif: mouse received primary T cells and intratumor injection of sortag; FIG. 9A and FIG. 9B are data collected from two independent replicates.

FIG. 10 shows the proliferation (FIG. 10A) and viability (FIG. 10B) of PNEscFv-CAR-T cells at 0 day (DO), 2 days (D2), 5 days (D5), 7 days (D7), and 10 days (D10) after lentivirus transduction. PNEscFv-CAR-T: primary T cells with PNEscfv-CAR expression; UN-T: primary T cells without PNEscFv-CAR expression.

FIG. 11A shows transduction efficiency and PNEscFv-CAR expression on the surface of T cells after 2 days (D2), 5 days (D5), 7 days (D7) and 10 days (D10) of transduction. The transduction efficiency was indicated by tagBFP. The PNEscFv-CAR expression on the surface of T cells was indicated by anti-biotin-PE, which was linked to CAR by Biotin-PNE-AALPETG*G (SEQ ID NO: 15).

FIG. 11B shows quantification of Biotin-PNE-AALPETG*G (SEQ ID NO: 15) sortagged on various solid tumor cell lines. SORT: Tumor cells were sortagged with Biotin-PNE-AALPETG*G (SEQ ID NO: 15); Blank: Tumor cells without sortagging. The sortagging efficiency was indicated by the intensity of anti-Biotin-PE.

FIG. 11C shows expression of CD25 and CD69 on the surface of T cells when co-cultured with various solid tumor cells. PNEscFv-CAR-T: primary T cells expressing PNEscFv-CAR; UN-T: primary T cell without PNEscFv-CAR expression; SORT: tumor cells sortagged with PNE-AALPETG*G (SEQ ID NO: 16); Blank: tumor cells without sortagging.

FIG. 12 shows cytokine release of T cells against tumor cells. PNEscFv-CAR-T: primary T cells expressing PNEscFv-CAR; UN-T: primary T cell without PNEscFv-CAR expression; the terms that end with “Blank” in x-axis labels mean tumor cells without sortagging; the terms that end with “SORT” in the x-axis labels mean tumor cells sortagged with PNE-AALPETG*G (SEQ ID NO: 16).

FIG. 13 shows cytotoxicity of PNEscFv-CAR-T cells against HCT-116, Hela or HepG2 solid tumor cell lines. Blank: tumor cells without sortagging; SORT: tumor cells sortagged with PNE-AALPETG*G (SEQ ID NO: 16).

FIG. 14 shows tumor size curves of mice receiving PNEscFv-CAR-T cells as well as sortag intratumor injections. The doses of sortag intratumor injections were limited to 2 times. The vertical arrows indicated the timepoints of giving one dose injection of the sortag mixtures to the mice. PNEscFv-CAR-T+Sortag: mouse received PNEscFv-CAR-T cells and intratumor injection of sortag; UN-T+Sortag: mouse received primary T cells and intratumor injection of sortag; Blank: mouse received PBS and no intratumor injection of sortag. The data were collected from four independent replicates.

FIG. 15 shows a Bi-CAR vector design for a PNE/CLDN18.2-CAR, which recognizes PNE via PNEscFv as well as Claudin 18.2 via CLDN18.2scFv. FIG. 15A shows that a Smart CAR modular design comprises PNE scFv, CLDN18.2 scFv, a hinge domain, a transmembrane domain, a co-stimulatory domain, and CD247. FIG. 15B, FIG. 15C, FIG. 15D, and FIG. 15E illustrate how the binding segments (e.g., a PNEscFv light chain, a PNEscFv heavy chain, a CLDN18.2scFv heavy chain, and a CLDN18.2scFv light chain) that recognize PNE and Claudin 18.2, respectively, can be arranged on a Bi-CAR design, wherein the binding segments are connected optionally by one or more linkers known in the art. FIG. 15B shows that the binding segments are arranged sequentially in the order of: CLDN18.2scFv heavy chain, PNEscFv light chain, PNEscFv heavy chain, and CLDN18.2scFv light chain. FIG. 15C shows that the binding segments are arranged sequentially in the order of: PNEscFv light chain, CLDN18.2scFv heavy chain, CLDN18.2scFv light chain, and PNEscFv heavy chain. FIG. 15D shows that the binding segments are arranged sequentially in the order of: PNEscFv light chain, PNEscFv heavy chain, CLDN18.2scFv heavy chain, and CLDN18.2scFv light chain. FIG. 15E shows that the binding segments are arranged sequentially in the order of: CLDN18.2scFv heavy chain, and CLDN18.2scFv light chain, PNEscFv light chain, and PNEscFv heavy chain.

FIG. 16 shows tumor size of mice receiving PNE/CLDN18.2-CAR-T (Bi-CAR-T) cells as well as sortag intratumor injection. In the tumor, only 10% tumor cells expressed antigen CLDN18.2. The vertical arrows indicated the timepoints of giving one dose injection of the respective sortag mixtures to the mice. PNE/CLDN18.2-CAR-T+Sortag: mouse received PNE/CLDN18.2-CAR-T cells and intratumor injection of sortag; UN-T+Sortag: mouse received primary T cells and intratumor injection of sortag. The data were collected from two independent experiment replicates.

FIG. 17 illustrates an example workflow for a cytokine releases assay and an activation assay. Primary T cells were transduced with lentivirus vectors and were engineered as Smart CAR-T cells (E: effector cells). Tumor cells were sortagged with exogenous antigens (T: tumor cells). The effector and tumor cells were co-cultured for 16 hours at the cell ratio of 2:1. Afterward, the supernatants were collected for cytokine release assay, and the cells were collected for activation assay by examining the expression of CD25 and CD69 on the effector cells.

FIG. 18 illustrates an example workflow for a cytotoxicity assay. Primary T cells were transduced with lentivirus vectors and were engineered as Smart CAR-T cells (E: effector cells). Tumor cells with stable luciferase expression were sortagged with exogenous antigens (T: tumor cells). The effector and tumor cells were co-cultured for 16 hours at series cell ratios. Afterward, luciferin was added to detect the cytotoxicity.

FIG. 19 illustrates an example workflow for an in vivo experiment testing a Smart CAR-T system described herein. Eight days (d-8) before the T cell injection, tumor cells were inoculated into mice subcutaneously (s.c.). One day (d-1) before the T cell injection, the mice were treated with radiation. At the d0 (day 0), T cells were injected into the mice intravenously (i. v.). In the next couple of days till dX (day X, for example, day 3, day 4 or day 5), a mixture of exogenous antigen and sortase were injected intratumor multiple times. The weight loss of the mice was monitored every three days after T cell injection till animal death or the tumor size reached the guideline of animal care ethical protocol.

FIG. 20 shows structures of certain modified peptides used herein. FIG. 20A shows the chemical structure of Biotin-AALPETG*G (SEQ ID NO: 14), in which G* refers to 2-hydroxyacetic acid and is boxed in a dashed rectangle. FIG. 20B shows the chemical structure of FITC-Ahx-GGGGK(Biotin)-NH2 (SEQ ID NO: 9), in which FITC-Ahx refers to fluorescein isothiocyanate with an aminohexanoic acid linker and is boxed in a dashed rectangle.

### DESCRIPTION

All publications cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

### DEFINITIONS

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein.

As used in this specification and the appended claims, the singular forms “a,” “an,” and “the” include plural referents unless the content clearly dictates otherwise. Thus, for example, reference to “a cell” includes one, two, or more cells, and the like.

The terms “polynucleotide” and “nucleic acid” are used interchangeably herein to refer to a polymeric form of nucleotides of any length, either ribonucleotides or deoxyribonucleotides. This term refers only to the primary structure of the molecule. A polynucleotide disclosed herein may be modified, e.g., with a labeling group such as a fluorophore, with a biotin, or with a phosphorothioate.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like. A peptide disclosed herein may be modified, e.g., with a labeling group such as a fluorophore, a biotin, a His tag, or phosphorothioate.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “subject” means any animal such as a mammal, e.g., a human.

The term “labeling,” “labeled,” “label” means that a detectable or identifiable group is attached to an entity, via covalent or non-covalent bond(s). For example, a protein, a nucleic acid, a polysaccharide can be labeled with a group such as a fluorophore, biotin, a His tag, or phosphorothioate. For another example, a cell may be labeled (also referred to as “conjugated,” “linked,” “anchored,” “ligated,” or “attached” herein) by a peptide facilitated by a sortase.

The term “sortagging,” “sortagged,” or “sortag” refers to sortase (e.g., Srt A)-mediated labeling of a cell either covalently or non-covalently. For example, any molecule that comprises a sortag motif, for example an LPXTG (SEQ ID NO: 2) (“X” refers to any amino acid) motif, can be labeled on a cell, mediated by a sortase, either covalently or non-covalently.

The term “sortag motif” as used herein refers to a stretch of amino acid sequence that can be recognized by a sortase. For example, a sortag motif may comprise LPXTG (“X” refers to any amino acid) (SEQ ID NO: 2), NP(Q/K)TN (SEQ ID NO: 3), (I/L)(P/A)XTG (e.g., LAXTG (SEQ ID NO: 7), IPXTG (SEQ ID NO: 17), IAXTG (SEQ ID NO: 18), LPXTG (SEQ ID NO: 2)), LPNTA (SEQ ID NO: 5), AALPETGXG (SEQ ID NO: 1), LAXTG (SEQ ID NO: 7), or the like. A sortag motif may include coded and non-coded amino acids, chemically or biochemically modified or derivatized amino acids, or a mimic of an amino acid. For example, a sortag motif AALPETG*G comprises a G*, which is 2-hydroxyacetic acid.

The term “tumor” as used herein refers to any abnormal and excessive growth of tissue in a subject. A tumor may be a soft tumor or solid tumor. A tumor may be benign, potentially malignant, or malignant. Non-limiting examples of tumors include lung cancer, gastric cancer, breast cancer, liver cancer, pancreatic cancer, colon cancer, kidney cancer, esophageal cancer, cervical cancer, and bladder cancer.

The term “antigen” is a molecule or molecular structure that can bind to another molecule or molecular structure such as a specific antibody or T-cell receptor, e.g., an engineered CAR. In other words, the “antigen” as used herein, is not limited to a substance that could stimulate antibody generation. In the Smart-CAR system disclosed herein, an antigen can be a molecule or molecular structure that does not necessarily stimulate antibody generation. For example, the antigen can be biotin, a peptide (such as PNE peptide NYHLENEVARLKKL (SEQ ID NO: 10), a chemical material (such as FITC, CY5), or the like.

The term “exogenous antigen ligand” as used herein refers to any molecule that does not typically express on the tissues of a subject. They include, but are not limited to, peptides such as a PNE peptide or leucin zipper, polysaccharides, and small molecules such as biotin, FITC, or TAMRA.

The term “endogenous antigen” as used herein refers to any molecule or molecular structure that typically expresses on the tissues of a subject. Such antigens include but are not limited to claudin8.2, Her2, Claudin6, GPC3, RGFR, MSLN, etc.

The term “exogenous antigen” refers to an exogenous antigen ligand fused with a sortag motif. An exogenous antigen may be directly fused with a sortag motif or via a spacer or a linker. For example, by linking a sortag motif such as a “AALPETGXG” (SEQ ID NO: 1) motif to an exogenous antigen ligand, e.g., to the C-terminus of an exogenous peptide antigen ligand, the exogenous antigen can be labeled onto tumor cells mediated by a sortase such as SrtA.

The term “binding domain” means any entity or component of an entity that is capable of binding an exogenous antigen ligand. Depending on the identity of the exogenous antigen ligand, for example, the binding domain may be a protein that binds the exogenous antigen ligand, or an antibody or functional fragments thereof that recognizes the exogenous antigen ligand. As another example, the exogenous antigen ligand may be a small molecule such as biotin, and the binding domain may be a binding partner, such as streptavidin, that recognizes the small molecule.

The term “chimeric antigen receptor” (CAR, also known as chimeric immunoreceptor, chimeric T cell receptor, or artificial T cell receptor) refers to a cell-surface receptor comprising an extracellular antigen binding domain, a transmembrane domain, and one or more cytoplasmic co-stimulatory signaling domains not naturally found together on a single receptor protein. A CAR may comprise an extracellular hinge region (e.g., a flexible spacer) between the extracellular antigen binding domain and the transmembrane domain. A CAR may combine an antigen-binding domain, a hinge domain, a transmembrane domain, a co-stimulatory domain, and CD247 into a single receptor. A “Smart CAR” as used herein refers to a CAR that comprises an extracellular antigen binding domain that recognizes an exogenous antigen ligand.

The term “immune cells” refer to cells of the immune system in animals, including neutrophils, cosinophils, basophils, mast cells, monocytes, macrophages, dendritic cells, natural killer cells, and lymphocytes (B cells and T cells). T cells comprise CD4+ cells, CD8+ cells, gamma delta T cells (γδ T cells), NK T cells, and/or regulatory T cells (Treg).

The term “CAR cells,” “engineered immune cells,” or “CAR-engineered immune cells” refer to immune cells that have been genetically engineered to express a chimeric antigen receptor or multiple (e.g., 2, 3, 4, etc.) chimeric antigen receptors (like Bi-CAR-T cells). The term “Smart CAR cells” as used herein refers to immune cells that have been genetically engineered to express Smart-CAR.

The term “CAR-T cells” refers to T cells that have been genetically engineered to express a chimeric antigen receptor or multiple chimeric antigen receptors (like Bi-CAR-T cells). T cells engineered with a CAR protein can have a new ability to target a specific antigen. The term “Smart Car-T cells” as used herein refers to CAR-T cells that have been genetically engineered to express a Smart CAR.

As used herein, “affinity” refers to the strength of the total of noncovalent interactions between a molecule or a molecular structure (e.g., a ligand) and its binding partner (e.g., a receptor). The affinity of a molecule for its partner can generally be represented by the equilibrium dissociation constant (Ko) (or its inverse equilibrium association constant, KA). Affinity can be measured by common methods known in the art, including those described herein. See, for example, Pope M. E., Soste M. V., Eyford B. A., Anderson N. L., Pearson T. W., (2009) J. Immunol. Methods. 341(1-2):86-96 and methods described therein.

As used herein, the term “specifically bind” or “bind” to, or “recognizes” a particular antigen refer to binding that is measurably different from a non-specific interaction. For example, in some embodiments, a binding molecule, such as a single domain antibody, specifically binds to a target molecule, such as an antigen, when the binding molecule reacts or associates more frequently, more rapidly, with greater duration, and/or with greater affinity with the particular target molecule than it does with alternative molecules. A binding molecule, such as a binding partner to an exogenous antigen, “specifically binds” to the exogenous antigen if it binds with greater affinity, avidity, more readily, and/or with greater duration than it binds to other molecules. It is understood that a binding molecule, such as the binding partner to an exogenous antigen, that specifically binds to a first target may or may not specifically bind to a second target. As such, “specific binding” does not necessarily require (although it can include) exclusive binding. In some embodiments, specific binding can be determined, for example, by comparing binding of a particular antibody to binding of an antibody that does not bind to a particular antigen. Specific binding for a particular antigen can be shown, for example, when a binder partner has a K) for an antigen of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, at least about 10⁻¹² M, or greater, where K_{D} refers to a dissociation rate of a binder partner/antigen interaction. In some embodiments, a binder partner that specifically binds nan antigen will have a K_{D} that is 20, 50, 100, 500, 1000, 5,000, 10,000 or more times greater than the K_{D} of a binder partner (or binding partner) that does not bind to the same antigen. In some embodiments, the binding between a binder partner and a particular antigen can be shown by an EC50 value, determined using suitable methods known in the art, including, for example, flow cytometry assay.

As used herein, a “composition” refers to any mixture of two or more products, substances, or compounds, including but not limited to, proteins, antibodies, polynucleotides, vectors, or cells. It may be a solution, a suspension, liquid, powder, a paste, aqueous, non-aqueous, or any combination thereof.

As used herein, a “pharmaceutical composition” refers to an active pharmaceutical agent formulated in pharmaceutically acceptable or physiologically acceptable solutions for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. Some non-limiting examples of the components that could be included in the composition are carriers, stabilizers, diluents, dispersing agents, suspending agents, thickening agents, and/or excipients. The pharmaceutical composition facilitates administration of the antibody or cell described herein to a subject. Multiple techniques of administration exist in the art including, but not limited to, intravenous, oral, aerosol, parenteral, ophthalmic, pulmonary, and topical administration.

As used herein, the term “pharmaceutically acceptable” refers to a material, such as a carrier or diluent, which does not abrogate the biological activity or properties of a therapeutic compound, and is relatively nontoxic, i.e., the material may be administered to a subject without causing undesirable biological effects or interacting in a deleterious manner with any of the components of the composition in which it is contained. Pharmaceutically acceptable components include those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “effective amount” or “therapeutically effective amount” refers to an amount of a pharmaceutical composition which is sufficient to significantly and positively modify the symptoms and/or conditions to be treated (e.g., provide a positive clinical response). The effective amount of a pharmaceutical composition will vary with the particular condition being treated, the severity of the condition, the duration of treatment, the nature of concurrent therapy, the particular composition being employed, the particular pharmaceutically-acceptable excipient(s) and/or carrier(s) utilized, and like factors with the knowledge and expertise of the attending physician.

As used herein, the term “treat,” “treating,” or “treatment” refers to ameliorating a disease or disorder, e.g., slowing or arresting or reducing the development of the disease or disorder or reducing at least one of the clinical symptoms thereof. For example, in some embodiments, ameliorating a disease or disorder can include obtaining a beneficial or desired clinical result that includes, but is not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting or eliminating the disease or progression of the disease, inhibiting or slowing the disease or its progression, arresting its development, and remission (whether partial or total).

### Smart CAR System and Methods of Use

The present disclosure provides a Smart CAR system, wherein an exogenous antigen is labeled on tumor cells, the tumor cells labeled with the exogenous antigen can be recognized by engineered immune cells that express a chimeric receptor comprising a binding domain that binds the exogenous antigen, and the tumor is treated, such as eliminated or at least partially eliminated.

The CAR-engineered immune cells can recognize the exogenous antigen labeled on the tumor cells, and may induce cytotoxicity to the tumor cells independent of their native tumor antigens. Therefore, the CAR-engineered immune cells can be applied as a universal anti-tumor treatment for tumors so long as the tumors are labeled with an antigen that is recognized by the CAR.

The labeling of the tumor cells can be realized by “sortagging” the tumor cells with an exogenous antigen. Sortagging tumor cells with an exogenous antigen may turn cold tumors into hot tumors through neo-antigen exposure caused by local cytotoxicity.

Any two molecules or molecular structures with specific binding affinity with each other can be used as an exogenous antigen ligand and the corresponding binding domain in a Smart CAR system, with one molecule (or molecular structure) expressed on the engineered immune cells as a pray and the other molecule (or molecular structure) labeled on the targeting tumor cells as a bait. For example, any antigen (e.g., biotin, PNE peptide, leucine zipper, or the like) that can be bound by an antibody or functional fragment, e.g., a single chain fragment variable (scFv) or that can be bound by a non-antibody binding partner, but that does not occur in the human proteome, can be used in a Smart-CAR system as an exogenous antigen.

In some embodiments, the present disclosure provides a method of treating a tumor in a subject in need thereof, comprising: (a) providing a sortase and an exogenous antigen to the tumor, wherein the exogenous antigen comprises an exogenous antigen ligand and a sortag motif, and wherein cells in the tumor are sortagged by the exogenous antigen, and (b) administering to the subject a therapeutically effective amount of genetically engineered immune cells that express a chimeric antigen receptor (CAR) comprising an antigen binding domain that is capable of binding the exogenous antigen.

In some embodiment, the method of treating a tumor further comprises administering to the subject genetically engineered immune cells that express at least one other CAR, wherein the one other CAR comprises a binding domain that recognizes an endogenous antigen or exogenous antigen in the tumor. In one embodiment, the method of treating a tumor further comprises administering to the subject genetically engineered immune cells that express one other CAR that recognizes the tumor, wherein the one other CAR recognizes an endogenous antigen in the tumor. Such CARs that recognize various endogenous antigens are known in the art. See, e.g., Nat Med. 2022 June; 28(6):1189-1198. Clin Cancer Res; 2020 Aug. 1; 26(15):3979-3989; Nat Med. 2021 September; 27(9):1544-1552.

In some embodiments, the present disclosure provides a method of treating a tumor in a subject in need thereof, comprising: (a) providing a sortase and an exogenous antigen to the tumor, wherein the exogenous antigen comprises an exogenous antigen ligand and a sortag motif, and wherein cells in the tumor are sortagged by the exogenous antigen, and (b) administering to the subject a pharmaceutical composition comprising a therapeutically effective amount of genetically engineered immune cells that express a chimeric antigen receptor (CAR) comprising an antigen binding domain that is capable of binding the exogenous antigen. Step (a) and step (b) in the methods as disclosed herein can be conducted around the same time or sequentially. Step (a) can be performed before step (b), or Step (b) can be performed before step (a). Step (a) can be performed multiple times, e.g., 1, 2, 3, 4, 5, 6, 7, 8, 9, and 10 times. Step (b) can be performed one time or multiple times such as 2 or 3 times. For example, step (a) can be conducted first on day 0, and then step (b) is conducted on day 0, and then step (a) is conducted for 5 additional times, on day 3, day 6, day 9, day 12, and day 15, respectively. Step (b) may be conducted on day 3 or day 6.

In some embodiments, the present disclosure provides a genetically engineered immune cell for treating a tumor, wherein a cell in the tumor is sortagged by an exogenous antigen mediated by a sortase, wherein the exogenous antigen comprises an exogenous antigen ligand and a sortag motif, and wherein the genetically engineered immune cell expresses a CAR comprising an antigen binding domain that is capable of binding the exogenous antigen.

In some embodiments, the present disclosure provides a pharmaceutical composition comprising a genetically engineered immune cell for treating a tumor, wherein a cell in the tumor is sortagged by an exogenous antigen mediated by a sortase, wherein the exogenous antigen comprises an exogenous antigen ligand and a sortag motif, and wherein the genetically engineered immune cell expresses a CAR comprising an antigen binding domain that is capable of binding the exogenous antigen.

In some embodiments, the present disclosure provides use of a genetically engineered immune cell for the manufacture of a medicament for treating a tumor, wherein a cell in the tumor is sortagged by an exogenous antigen mediated by a sortase, wherein the exogenous antigen comprises an exogenous antigen ligand and a sortag motif, and wherein the genetically engineered immune cell expresses a CAR comprising an antigen binding domain that is capable of binding the exogenous antigen.

In some embodiments, the present disclosure provides use of a pharmaceutical composition comprising a genetically engineered immune cell for the manufacture of a medicament for treating a tumor, wherein a cell in the tumor is sortagged by an exogenous antigen mediated by a sortase, wherein the exogenous antigen comprises an exogenous antigen ligand and a sortag motif, and wherein the genetically engineered immune cell expresses a CAR comprising an antigen binding domain that is capable of binding the exogenous antigen.

In some embodiment, the CAR comprising an antigen binding domain that is capable of binding the exogenous antigen ligand further comprises at least one other binding domain that is capable of binding at least one other antigen in the tumor that is endogenous or exogenous. For example, the CAR comprising an antigen binding domain that is capable of binding the exogenous antigen ligand can further comprise one, two, three, or more additional binding domains that is capable of binding one, two, three, or more additional exogenous or endogenous antigen in the tumor.

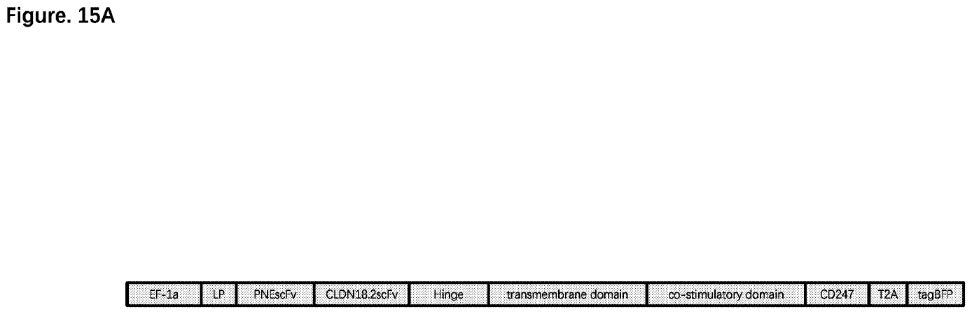

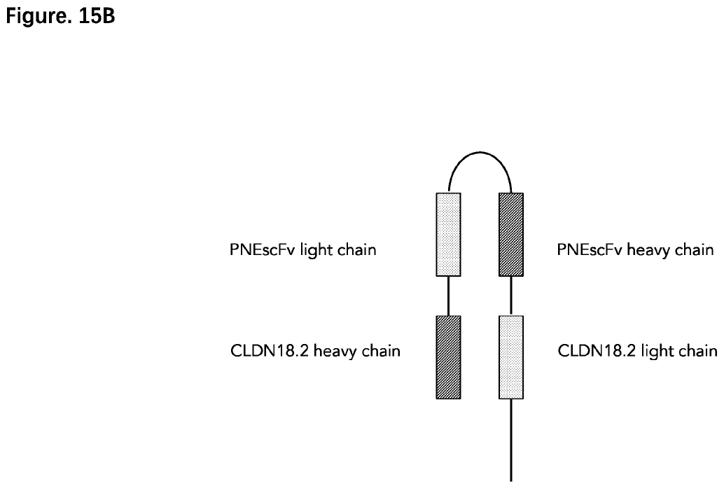

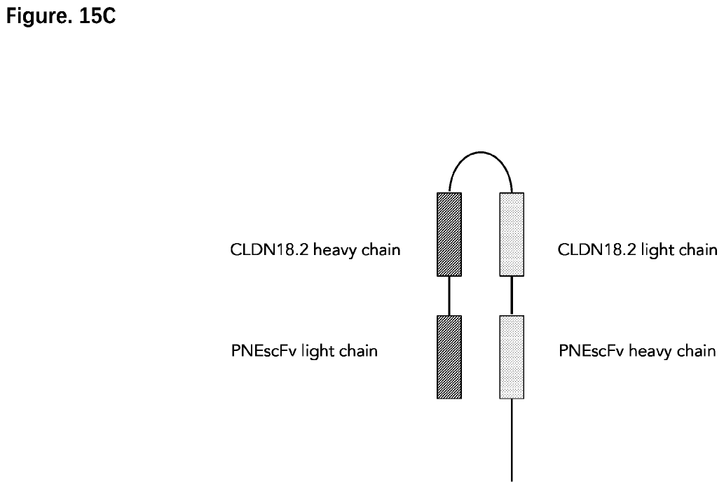

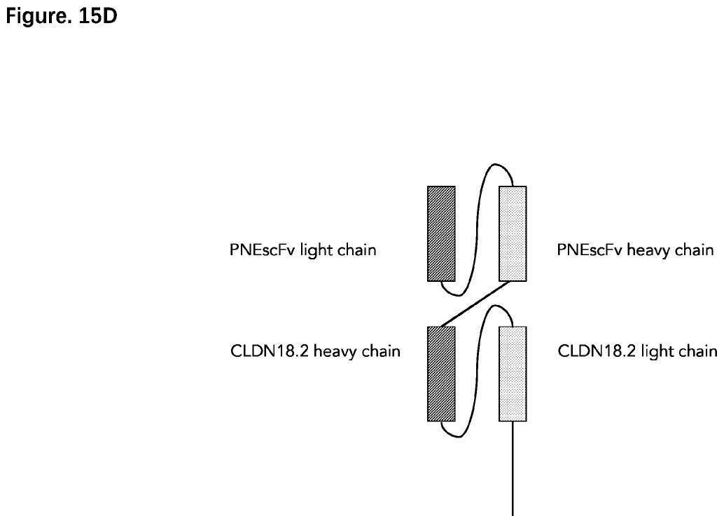

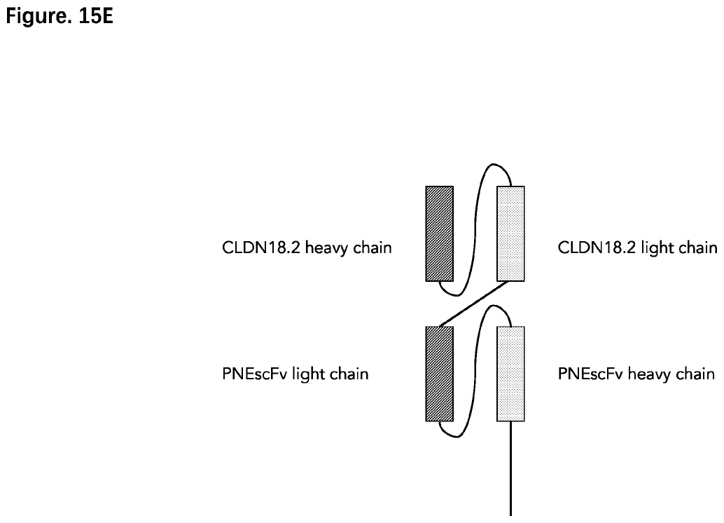

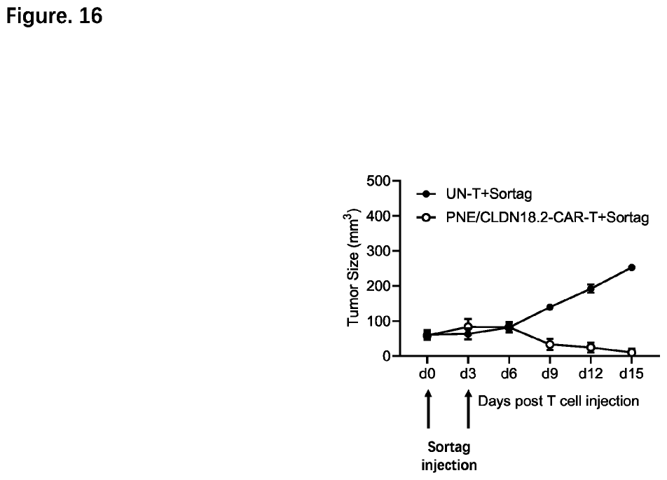

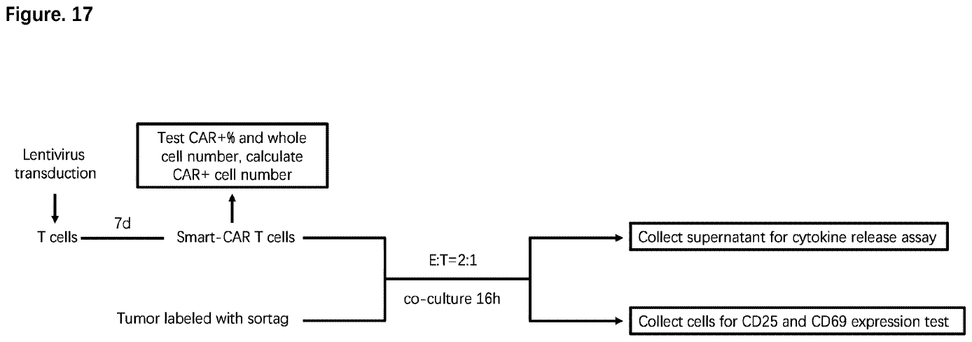

In some embodiment, the CAR comprising an antigen binding domain that is capable of binding the exogenous antigen ligand further comprises a second binding domain that is capable of binding an endogenous antigen in the tumor. For example, FIG. 17 illustrates a CAR that comprises two binding domains: a first binding domain such as PNEscFv (anti-PNE single chain variable fragment) and a second binding domain such as CLDN18.2scFv (anti-CLDN18.2 single chain variable fragment). The first and second binding domains may be connected optionally by a hinge know in the art. As illustrated in FIGS. 15A-15E, the binding segments of the binding domains can have alternative arrangements so long as the binding domains can achieve the purpose of recognizing both the exogenous antigen and the endogenous antigen. The PNEscFv can recognize a tumor cell that is sortagged with PNE and the CLDN18.2scFv can recognize an endogenous antigen claudin8.2 on the tumor cell. Such a dual-CAR (or Bi-CAR) system can enhance or provide synergistic effects of tumor killing. As discussed in Example 3 and FIG. 16, we designed a Bi-CAR system, wherein two CARs were expressed on each T Cell. One CAR was PNE-CAR, which recognized PNE-AALPETG*G labeled on tumor cells by sortase; the other CAR was a clinically used CAR, which recognized the antigen claudin8.2 (CLDN18.2) of stomach cancer. We established a low antigen expressing tumor model, in which only 10% tumors express CLDN18.2. Usually, this low antigen expressing tumor could not be effectively eliminated by CAR-T cells. But when a sortag mixture and Bi-CAR-T cells were used in the animal model, we found the low antigen expressing tumor could be significantly controlled.

Binding domains (such as antibodies, e.g., scFv) that recognize endogenous antigens are readily available based on the art. See, e.g., Biomark Res. 2022 May 31; 10(1):38; Front Oncol. 2022 Feb. 16; 12:824208; Nat Rev Clin Oncol. 2020 January; 17(1):33-48; Semin Immunol. 2020 February; 47:101391.

In some embodiments, at least some of the tumor cells sortagged by an exogenous antigen are eliminated by the Smart CAR cells administered to a subject. In some embodiments, tumor cells in the vicinity to the tumor cells sortagged by an exogenous antigen are also eliminated, at least partially, by the Smart CAR cells. In some embodiments, treatment with Smart CAR cells changes the tumor environment and boost the endogenous immunity against tumors to improve the long-term survival of patients.

In some embodiments, the tumor is a solid tumor.

In some embodiments, the tumor is selected from Lung cancer, gastric cancer, breast cancer, liver cancer, pancreatic cancer, colon and rectal cancer, kidney cancer, esophageal cancer, cervical cancer, bladder cancer, endometrial cancer, and thyroid cancer.

In some embodiments, the sortase is selected from sortase A, sortase B, and variants thereof. In some embodiments, the sortase is SrtA. In some embodiments, the sortase is mgSrtA.

In some embodiments, the exogenous antigen ligand is a peptide such as a PNE peptide or leucine zipper, or a small molecule such as a molecular label, e.g., biotin, FITC, or TAMRA.

In some embodiments, the sortag motif is selected from LPXTG (SEQ ID NO: 2), NP(Q/K)TN (SEQ ID NO: 3), (I/L)(P/A)XTG (e.g., LAXTG (SEQ ID NO: 7), IPXTG (SEQ ID NO: 17), IAXTG (SEQ ID NO: 18), LPXTG (SEQ ID NO: 2)), LPNTA (SEQ ID NO: 5), AALPETGXG (SEQ ID NO: 1), and LAXTG (SEQ ID NO: 7).

In some embodiments, the sortase and the exogenous antigen are provided to the tumor sequentially, e.g., within a few minutes apart. In some embodiments, the sortase is provided to the tumor first, and then the exogenous antigen is provided to the tumor. In some embodiments, the exogenous antigen is provided to the tumor first, and then the sortase is provided to the tumor. In some embodiments, a pharmaceutical acceptable composition comprising a sortase is provided to the tumor first, and then a pharmaceutical acceptable composition comprising an exogenous antigen is provided to the tumor. In some embodiments, a pharmaceutical acceptable composition comprising an exogenous antigen is provided to the tumor first, and then a pharmaceutical acceptable composition comprising a sortase is provided to the tumor.

In some embodiments, a mixture of the sortase and the exogenous antigen are provided to the tumor.

In some embodiments, a pharmaceutical acceptable composition comprising a mixture of the sortase and the exogenous antigen are provided to the tumor.

In some embodiments, in step (a) of the methods as disclosed herein, the sortase and the exogenous antigen are provided to the subject in the same composition (e.g., a mixture), and in some embodiments, provided in separate compositions. When in separate compositions, the sortase and the exogenous antigen can be provided to the subject around the same time or sequentially.

In some embodiments, the sortase and the exogenous antigen are provided to the tumor or the vicinity of the tumor by injection.

In some embodiments, the sortase and the exogenous antigen are provided to the tumor by intratumor injection. For example, intratumoral administration using imaging-guided injection can be accomplished in most organs (8). Intratumoral immunotherapies in clinical applications can increase in situ bioavailability and the efficiency of immunotherapy (9, 10).

In some embodiments, the sortase is provided to the tumor in an amount of 1 to 1000 μM, e.g., 20 to 800 μM. e.g., 400 to 500 μM.

In some embodiments, the exogenous antigen is provided to the tumor in an amount of 1 to 1000 M, e.g., 20 to 800 μM, e.g., 400 to 500 M.

In some embodiments, the subject is a mammal. In a preferred embodiment, the subject is a human.

In some embodiments, the CAR comprises an antigen binding domain, a transmembrane domain, one or more costimulatory signaling regions, and a cytoplasmic domain comprising an immunoreceptor tyrosine-based activation motif (ITAM), wherein the antigen binding domain is capable of binding an exogenous antigen.

In some embodiments, the cytoplasmic domain comprises a CD247 cytoplasmic domain.

In some embodiments, the one or more costimulatory signaling regions are selected from CD28, CD27, CD134 (OX40), and CD137 (4-1BB).

In some embodiments, a chimeric antigen receptor further comprises one or more extracellular leader domains and/or one or more extracellular hinge domains.

In some embodiments, the antigen binding domain is a scFv that is capable of binding an exogenous antigen.

In some embodiments, the Smart CAR cells are engineered immune cells, such as lymphocytes, e.g., T cells. In some embodiments, the Smart CAR cells are engineered immune cells such as T cells, NK cells, macrophages, dendritic cells, or B cells. In a preferred embodiment, the Smart CAR cells are Smart CAR-T cells.

In some embodiments, the immune cells are harvested from a subject in need thereof before genetically engineered with the CAR. In some embodiments, the immune cells are harvested from a subject that is not from the subject having a tumor. In a preferred embodiment, T cells are harvested from the subject in need thereof before genetically engineered with the CAR.

The therapeutically effective amount of the engineered immune cells, e.g., engineered T cells, can be determined based on factors of a particular subject, including size, age, sex, weight, and condition of the subject. Dosages can be ascertained and adjusted by those skilled in the art from this disclosure and the knowledge in the art.

In some embodiments, the engineered immune cells, e.g., engineered T cells, can be administered in any pharmaceutically acceptable vehicle. In some embodiments, a therapeutically effective amount of about 1×10⁵ to 1×10¹², 1×10⁶ to 1×10¹¹, 1×10⁶ to 1×10¹⁰, 1×10⁶ to 1×10⁹, 1×10⁷ to 1×10¹¹, 1×10⁷ to 1×10¹⁰, 1×10⁷ to 1×10⁹, or 1×10⁸ to 1×10⁹ cells are administered. In some embodiments, a dose of about 1×10⁶, 1×10⁷, 1×10⁸, 1×10⁹, 1×10¹⁰ or 1×10¹¹ cells are administered to a subject having tumor. In some embodiments, the engineered T cells, can be administered in one dose or two doses.

In some embodiments, the purity in populations comprising the engineered immune cells, e.g., engineered T cells, ranges from about 70% to about 75%, about 75% to about 80%, about 80% to about 85%, preferably about 85% to about 90%, about 90% to about 95%, and about 95% to about 100%. For example, the present disclosure provides a population of T cells wherein 70% to 80% of the T cells are engineered T cells and 20% to 30% of the T cells are non-engineered T cells.

The engineered immune cells, e.g., engineered T cells, can be administered by, for example, injection or catheter, e.g., by intravenous injection or infusion. The engineered immune cells, e.g., engineered T cells, may also be administered by minimally invasive surgical techniques.

In some embodiments, the engineered immune cells, e.g., engineered T cells, are administered to a subject having tumor, in a predetermined time after a sortase and an exogenous antigen are provided to the tumor. For example, the predetermined time can range from 1 hour to 8 hours, 2 hours to 8 hours, 3 hours to 7 hours, 3 hours to 6 hours, 3 hours to 5 hours, or the like.

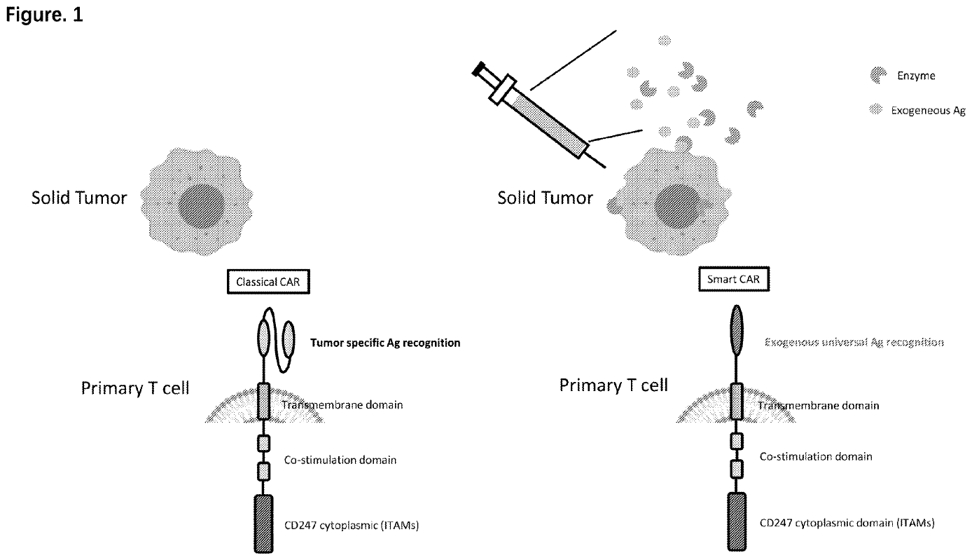

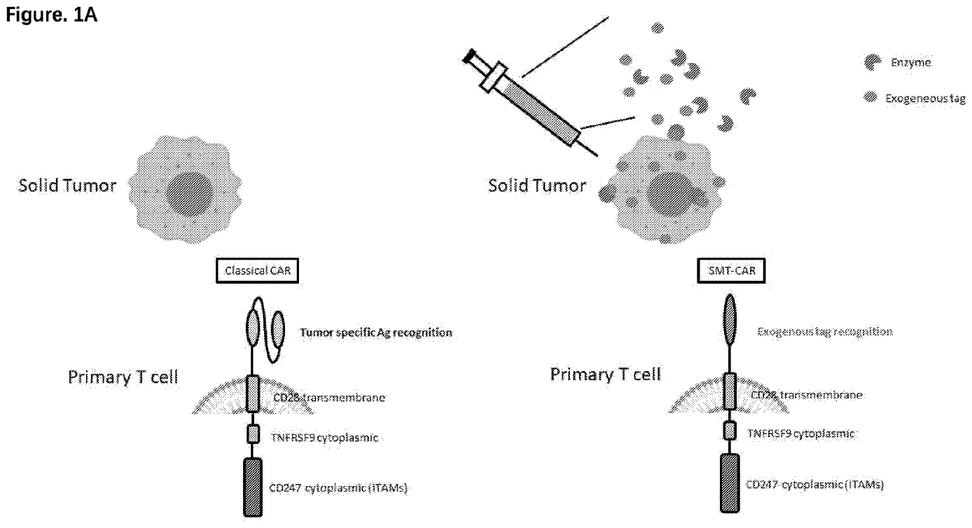

In some embodiments, the present disclosure provides a Smart CAR-T system for treating a tumor, as illustrated in FIG. 1:
a sortase (denoted as “Enzyme”) and an exogenous antigen (denoted as “Exogenous Ag”) are delivered to a solid tumor via injection, e.g., intratumor injection;
the exogenous antigen is labeled on the tumor mediated by the enzyme within several hours; Smart CAR that recognizes the exogenous antigen is stably expressed on primary T cells (Smart CAR-T cells);
Smart CAR-T cells are delivered to a subject having a tumor, and
the Smart CAR-T cells can be activated by the tumor labeled with exogenous antigen.
   This CAR-T system can label tumors with exogenous antigens efficiently and enable tumor elimination by CAR-T cells effectively.

A Smart CAR system, e.g., Smart CAR-T system, as described herein can achieve one or more of the following advantages. The Smart CAR system, e.g., Smart CAR-T system, can induce cytotoxicity to the tumor cells independent of their native tumor antigens. The Smart CAR system, e.g., Smart CAR-T system, bypasses the challenge of screening for tumor antigens or scFvs that specifically bind tumor antigens. The tumor antigen-independent working manner prevents the Smart CAR cells (e.g., Smart CAR-T cells) from acquired resistance caused by heterogeneity and antigen loss in the tumor. The tumor cytotoxicity induced by the Smart CAR cells (e.g., Smart CAR-T cells) can help to expose neoantigen to primary T cells. The introduction of exogenous antigen can weaken the immune escape caused by tumor mutation.

The Smart CAR system and methods can also be used in a combination with one or more other treatments, sequentially or concomitantly, that is suitable for treating a tumor. For example, the Smart CAR system and methods can also be used in a combination with, e.g., chemotherapy, cytokines, growth factors, hormones, pro-drugs, drugs, antibodies, or other various pharmaceutically active agents.

### Tumor Cell Labeling

In some embodiments, the present disclosure provides a method of labeling a tumor cell with an exogenous antigen, comprising providing a sortase and an exogenous antigen to the tumor cell, wherein the exogenous antigen comprises an exogenous antigen ligand and a sortag motif, and wherein the tumor cell is sortagged by the exogenous antigen.

In some embodiments, the sortase is selected from sortase A, sortase B, and variants thereof. In some embodiments, the sortase is mgSrtA.

In some embodiments, wherein the exogenous antigen ligand is a peptide or a molecular label.

In some embodiments, the sortag motif is selected from LPXTG (SEQ ID NO: 2), NP(Q/K)TN (SEQ ID NO: 3), (I/L)(P/A)XTG (e.g., LAXTG (SEQ ID NO: 7), IPXTG (SEQ ID NO: 17), IAXTG (SEQ ID NO: 18), LPXTG (SEQ ID NO: 2)), LPNTA (SEQ ID NO: 5), AALPETGXG (SEQ ID NO: 1), and LAXTG (SEQ ID NO: 7).

In some embodiments, the tumor cell is selected from primary cells and immortalized cells.

In some embodiments, the sortase and the exogenous antigen are injected to the tumor cell or the vicinity of the tumor cell.

In some embodiments, the method of labeling occurs in vivo in a subject.

In some embodiments, the method of labeling occurs in vitro.

In some embodiments, the sortase and the exogenous antigen are provided to the tumor cell sequentially.

In some embodiments, a mixture of the sortase and the exogenous antigen are provided to the tumor cell.

In some embodiments, the present disclosure provides a tumor cell sortagged with an exogenous antigen by a method of labeling tumor cells described herein.

In some embodiments, the present disclosure provides a tumor cell sortagged with an exogenous antigen.

In some embodiments, the exogenous antigen is conjugated to the plasma membrane of the tumor cell.

In some embodiments, the present disclosure provides an exogenous antigen comprising a sortag motif and an exogenous antigen ligand.

In some embodiments, the exogenous antigen ligand is a peptide or a molecular label.

### Smart CAR Kits

In some embodiments, the present disclosure provides a Smart CAR kit, comprising an exogenous antigen and a sortase. In some embodiments, the kit comprises a mixture of an exogenous antigen and sortase.

In some embodiments, the sortase is selected from sortase A, sortase B, and variants thereof. In some embodiments, the sortase is mgSrtA.

In some embodiments, the Smart CAR kit further comprises a vector comprising a nucleic acid encoding a chimeric antigen receptor (CAR), wherein the CAR comprises an antigen binding domain, a transmembrane domain, one or more costimulatory signaling region, and a cytoplasmic domain comprising an immunoreceptor tyrosine-based activation motif (ITAM), wherein the antigen binding domain is capable of binding an exogenous antigen described herein.

In some embodiment, the CAR comprising an antigen binding domain that is capable of binding the exogenous antigen ligand further comprises at least one other binding domain that is capable of binding at least one other antigen in the tumor that is endogenous or exogenous.

In some embodiment, the CAR comprising an antigen binding domain that is capable of binding the exogenous antigen ligand further comprises a second binding domain that is capable of binding an endogenous antigen in the tumor.

### Sortases

The sortase as used herein can be any naturally occurring sortase or functional variant thereof. There are at least six classes of Sortases, including Sortase Class A, B, C, D, E, and F, as shown in the table below [12].

TABLE 1
Sortase classes, substrates and substrate
recognition motifs with species specificity

| Sortase | | | |
|---|---|---|---|
| class | Motif | Substrates | Species |
| A | LPXTG | Surface proteins | All low GC content |
| | | | Gram-positive bacteria |
| B | NP(Q/K)TN | Haem | Low GC content |
| | | acquisition | Gram-positive |
| | | proteins | bacilli and cocci |
| C | (I/L)(P/A)XTG | Pilin subunits | Both low and high |
| | | | GC content |
| | | | Gram-positive bacteria |
| D | LPNTA | Endospore | Bacillus species |
| | | envelope | |
| | | proteins | |
| E | LAXTG | Pili | High GC content |
| | | | Gram-positive bacteria |
| F | — | — | Actinobacteria |

As noted above, a diverse range of sortase variants have been developed, including a sortase variant (eSrtA, 5M) [13], Srt7M [14], the Chen group's evolved variant based on the 5M variant [15], the Chen group's “promiscuous” SrtA variant, mgSrtA [16], and an LMVGG-recognizing SrtA variant [17].

In some embodiments, a sortase, selected from Sortase Class A, B, C, D, E, and F and any variants thereof, is used for labeling a tumor cell with an exogenous antigen. In some embodiments, a sortase, selected from SrtA and any variants thereof, is used for labeling a tumor cell with an exogenous antigen. In some embodiments, a sortase, selected from SrtA, mgSrtA, 5M, and Srt7M, is used for labeling a tumor cell with an exogenous antigen.

### CAR and Engineered Immune Cells

In some embodiments, the present disclosure provides a nucleic acid encoding a chimeric antigen receptor (CAR), wherein the CAR comprises an antigen binding domain, a transmembrane domain, one or more costimulatory signaling region, and a cytoplasmic domain comprising an immunoreceptor tyrosine-based activation motif (ITAM), wherein the antigen binding domain is capable of binding an exogenous antigen described herein.

In some embodiment, the CAR comprising an antigen binding domain that is capable of binding the exogenous antigen ligand further comprises at least one other binding domain that is capable of binding at least one other antigen in the tumor that is endogenous or exogenous.

In some embodiment, the CAR comprising an antigen binding domain that is capable of binding the exogenous antigen ligand further comprises a second binding domain that is capable of binding an endogenous antigen in the tumor.

In some embodiments, the cytoplasmic domain comprises a CD247 cytoplasmic domain.

In some embodiments, the one or more costimulatory signaling regions are selected from CD28, CD27, CD134 (OX40), and CD137 (4-1BB).

In some embodiments, a chimeric antigen receptor further comprises one or more extracellular leader domains and/or one or more extracellular hinge domains. In some embodiments, one or more extracellular hinge domains comprise a CD28 extracellular hinge domain, CD8a extracellular hinge domain, or an IgG4 extracellular hinge domain.

In some embodiments, a transmembrane domain comprises a CD28, CD8a, CD64, CD32a, CD32c, CD16a, TRL1, TLR2, TLR3, TRL4, TLR5, TLR6, TLR7, TLR8, or TLR9 transmembrane domain.

In some embodiments, the antigen binding domain is an scFv that is capable of binding the exogenous antigen.

In some embodiments, the present disclosure provides a vector comprising a nucleic acid described herein. Nonlimiting examples of vectors include retrovirus vectors, AAV vectors, and oncolytic virus vectors. In some embodiments, the vector is or comprises a lentiviral vector.

In some embodiments, the disclosure provides engineered immune cells that express a CAR comprising an antigen binding domain, a transmembrane domain, one or more costimulatory signaling region, and a cytoplasmic domain comprising an ITAM, wherein the antigen binding domain is capable of binding an exogenous antigen described herein.

In some embodiments, the disclosure provides engineered immune cells comprising a nucleic acid or a vector that encodes a CAR comprising an antigen binding domain, a transmembrane domain, one or more costimulatory signaling region, and a cytoplasmic domain comprising an ITAM, wherein the antigen binding domain is capable of binding an exogenous antigen described herein.

In some embodiments, the present disclosure provides a method of modifying an immune cell comprising delivering to an immune cell a vector or a nucleic acid described herein.

In some embodiments, the immune cells are selected from a macrophage, monocyte, dendritic cell, T cells, B cells and/or NK cells. In some embodiments, the immune cells are T cells, NK cells, macrophages, dendritic cells, or B cells. In some embodiments, the immune cells are T cells. In some embodiments, the T cells are selected from CD4+ cells, CD8+ cells, Gamma delta T cells (γδ T cells), NK T cells and/or regulatory T cells (Treg).

In some embodiments, the immune cells are immune cells harvested from a human subject having tumor. In some embodiments, the immune cells are T cells harvested from a human subject having tumor.

For example, white blood cells from a subject diagnosed with tumor are harvested, T cells are isolated and cultured, and the T cells are transformed with a vector containing a nucleic acid encoding a CAR described herein. T cells prepared for administration to a subject can comprise a purified population of cells, for example CD4+ T cells. Those having ordinary skill in the art can readily determine the percentage of genetically modified immune cells in a population using various well-known methods, such as fluorescence activated cell sorting (FACS).

### Pharmaceutical Compositions

In some embodiments, the disclosure provides pharmaceutical compositions comprising an immune cell described herein. In some embodiments, a pharmaceutical composition comprises a pharmaceutically acceptable carrier.

In some embodiments, the disclosure provides pharmaceutical compositions comprising a sortase described herein. In some embodiments, a pharmaceutical composition comprises a pharmaceutically acceptable carrier.

In some embodiments, the disclosure provides pharmaceutical compositions comprising an exogenous antigen described herein. In some embodiments, a pharmaceutical composition comprises a pharmaceutically acceptable carrier.

In some embodiments, the disclosure provides pharmaceutical compositions comprising a sortase and an exogenous antigen described herein. In some embodiments, a pharmaceutical composition comprises a pharmaceutically acceptable carrier.

In some embodiments, pharmaceutical compositions comprising engineered immune cells, e.g., CAR-T cells, are provided as sterile liquid preparations, e.g., isotonic aqueous solutions, suspensions, emulsions, dispersions, or viscous compositions, which may be buffered to a selected pH. In some embodiments, pharmaceutically acceptable carrier is selected from water, saline, phosphate buffered saline, polyol (for example, glycerol, propylene glycol, liquid polyethylene glycol, and the like) and suitable mixtures thereof.

In some embodiments, the disclosure provides pharmaceutical compositions comprising a nucleic acid or a vector described herein. In some embodiments, a pharmaceutical composition comprises a pharmaceutically acceptable carrier.

### EXAMPLES

The following examples are provided to describe the disclosure in greater detail. They are intended to illustrate, not to limit, the disclosure. The results reported in Examples 1 and 2, along with the experimental procedures described in Examples 3-13, illustrate that the Smart CAR-T system as disclosed herein can effectively target and treat tumors both in vitro and in vivo.

### Example 1: mSA-Smart CAR-T System

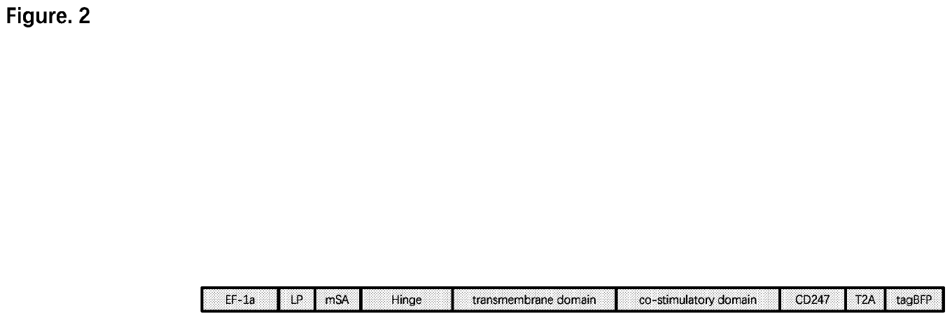

In an mSA-Smart CAR-T system, tumor cells were labeled by an exogeneous antigen comprising biotin and a sortase motif, and the CAR-T cells (referred to as “mSA-CAR-T cells”) were engineered to express a CAR comprising an affinity-enhanced monomeric streptavidin (mSA) biotin-binding domain, which is capable of recognizing and binding to biotin. FIG. 2 illustrates a vector design for a Smart CAR module wherein an affinity-enhanced monomeric streptavidin (mSA) biotin-binding domain was merged with components from a canonical CAR including a hinge domain, a transmembrane domain, a co-stimulatory domain, and CD247. A tagBFP (blue fluorescence protein) marker was included to mark the cells transduced by the Smart CAR vector (FIG. 2). tagBFP is a basic (constitutively fluorescent) blue fluorescent protein derived from Entacmaea quadricolor.

SEQ ID NO: 11 provides a vector encoding a Smart CAR system:

(SEQ ID NO: 11)
GGCTCCGGTGCCCGTCAGTGGGCAGAGCGCACATCGCCCACAGTCCCCGAGAAGTT
GGGGGGAGGGGTCGGCAATTGAACCGGTGCCTAGAGAAGGTGGCGCGGGGTAAAC
TGGGAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATGGC
CCTTGCGTGCCTTGAATTACTTCCACCTGGCTGCAGTACGTGATTCTTGATCCCGAGC
TTCGGGTTGGAAGTGGGTGGGAGAGTTCGAGGCCTTGCGCTTAAGGAGCCCCTTCGC
CTCGTGCTTGAGTTGAGGCCTGGCCTGGGCGCTGGGGCCGCCGCGTGCGAATCTGGT
GGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGA
TCTGCACACTGGTATTTCGGTTTTTGGGGCCGCGGGCGGCGACGGGGCCCGTGCGTC
CCAGCGCACATGTTCGGCGAGGCGGGGCCTGCGAGCGCGGCCACCGAGAATCGGAC
GGGGGTAGTCTCAAGCTGGCCGGCCTGCTCTGGTGCCTGGCCTCGCGCCGCCGTGTA
TCGCCCCGCCCTGGGCGGCAAGGCTGGCCCGGTCGGCACCAGTTGCGTGAGCGGAA
AGATGGCCGCTTCCCGGCCCTGCTGCAGGGAGCTCAAAATGGAGGACGCGGCGCTC
GGGAGAGCGGGCGGGTGAGTCACCCACACAAAGGAAAAGGGCCTTTCCGTCCTCAG
CCGTCGCTTCATGTGACTCCACGGAGTACCGGGCGCCGTCCAGGCACCTCGATTAGT
TCTCGAGCTTTTGGAGTACGTCGTCTTTAGGTTGGGGGGAGGGGTTTTATGCGATGG
AGTTTCCCCACACTGAGTGGGTGGAGACTGAAGTTAGGCCAGCTTGGCACTTGATGT
AATTCTCCTTGGAATTTGCCCTTTTTGAGTTTGGATCTTGGTTCATTCTCAAGCCTCA
GACAGTGGTTCAAAGTTTTTTTCTTCCATTTCAGGTGTCGTGATCTAGAGCGCTGCCA
CCATGGAGACAGACACACTCCTGCTATGGGTGCTGCTGCTCTGGGTTCCAGGTTCCA
CAGGTGACGGCGCCGAGGCTGGCATCACCGGTACCTGGTACAACCAGCATGGATCG
ACCTTTACTGTCACTGCCGGCGCGGACGGCAACCTGACCGGCCAGTATGAGAACCG
CGCTCAGGGTACTGGGTGCCAGAACAGCCCTTACACCCTCACTGGCCGCTACAACG
GCACCAAGCTGGAGTGGCGGGTGGAGTGGAACAACTCCACGGAGAATTGTCACAGC
CGCACCGAGTGGCGTGGTCAATACCAGGGGGGGGCAGAGGCCAGGATTAATACCCA
GTGGAACTTGACCTACGAGGGCGGCTCCGGGCCGGCCACAGAACAGGGACAGGATA
CGTTCACCAAAGTGAAGCCCTCTGCGGCTTCCGGATCTACCGTGTCCTCGGAGTCCA
AGTACGGCCCTCCCTGTCCCCCGTGCCCAATCTACATATGGGCACCGCTGGCCGGCA
CCTGTGGTGTCCTGTTGCTATCTCTGGTGATCACACTGTACTGTAAGCGCGGCCGCA
AGAAGCTGCTGTATATCTTCAAGCAGCCCTTCATGAGACCGGTGCAGACGACTCAGG
AGGAAGATGGGTGCTCTTGTAGGTTTCCTGAGGAGGAGGAGGGCGGGTGCGAGCTG
CGCGTAAAATTTTCACGCAGCGCGGACGCACCCGCCTACAAGCAAGGACAGAACCA
GCTCTACAACGAGCTCAACCTGGGCCGGCGTGAAGAGTACGATGTGCTGGACAAGC
GTCGCGGCCGCGACCCGGAGATGGGCGGCAAGCCGCGGCGTAAGAATCCACAGGA
GGGGCTTTACAACGAGTTGCAGAAGGACAAAATGGCCGAGGCCTACTCCGAGATCG
GTATGAAGGGCGAGCGCCGTCGTGGTAAAGGGCATGACGGCCTTTATCAGGGGCTG
TCCACGGCCACGAAAGACACCTACGACGCGCTGCATATGCAGGCCCTTCCTCCTCGA
TGTACAGGCAGTGGAGAGGGCAGGGGAAGTCTGCTAACATGCGGGGACGTGGAGG
AAAATCCCGGCCCCGGCAGCGAGCTGATTAAGGAGAACATGCACATGAAGCTGTAC
ATGGAGGGCACCGTGGACAACCATCACTTCAAGTGCACATCCGAGGGCGAAGGCAA
GCCCTACGAGGGCACCCAGACCATGAGAATCAAGGTGGTCGAGGGCGGCCCTCTCC
CCTTCGCCTTCGACATCCTGGCTACTAGCTTCCTCTACGGCAGCAAGACCTTCATCA
ACCACACCCAGGGCATCCCCGACTTCTTCAAGCAGTCCTTCCCTGAGGGCTTCACAT
GGGAGAGAGTCACCACATACGAGGACGGGGGCGTGCTGACCGCTACCCAGGACACC
AGCCTCCAGGACGGCTGCCTCATCTACAACGTCAAGATCAGAGGGGTGAACTTCAC
ATCCAACGGCCCTGTGATGCAGAAGAAAACACTCGGCTGGGAGGCCTTCACCGAGA
CCCTGTACCCCGCTGACGGCGGCCTGGAAGGCAGAAACGACATGGCCCTGAAGCTC
GTGGGCGGGAGCCATCTGATCGCAAACATCAAGACCACATATAGATCCAAGAAACC
CGCTAAGAACCTCAAGATGCCTGGCGTCTACTATGTGGACTACAGACTGGAAAGAA
TCAAGGAGGCCAACAACGAGACCTACGTCGAGCAGCACGAGGTGGCAGTGGCCAG
ATACTGCGACCTCCCTAGCAAACTGGGGCACAAGCTTAATTAA

As illustrated in FIG. 2 and shown in the sequence of SEQ ID NO: 11, the nucleotides 1-1179 in SEQ ID NO: 11 represents EF-1a, the nucleotides 1197-1259 encodes LP (leading peptide), the nucleotides 1260-1625 encodes mSA, the nucleotides 1638-1673 encodes Hinge, the nucleotides 1674-1745 encodes Transmembrane domain, the nucleotides 1746-1871 encodes co-stimulatory domain, the nucleotides 1872-2207 encodes CD247, the nucleotides 2223-2276 encodes T2A, the nucleotides 2277-2975 encodes tagBFP. Alternative nucleotide codons can be used to generate the Smart CAR system.

As illustrated in FIG. 2, the Smart CAR vector also included the human elongation factor 1 alpha (EF-1 alpha) first intron, which is capable of highly enhancing expression of foreign genes in mammalian cells.

As illustrated in FIG. 2, the Smart CAR vector comprises a component encoding a leading peptide (LP), which typically has a sequence of 16 to 20 amino acids at the N-terminus of some eukaryotic proteins that determines their ultimate destination. Proteins that are made and function in the cytosol lack a leading peptide. Proteins destined for specific organelles require signal sequences appropriate for each organelle. The leading peptide for a protein destined to enter the endoplasmic reticulum typically contains hydrophobic amino acids that become embedded in the lipid bilayer membrane, and it functions to guide the nascent protein to a receptor protein that marks the position of a pore in the membrane. Once the protein passes into the cysternal lumen through the pore, the leading peptide is cleaved from the protein. In plasmid designs, the leading peptide is typically located between the promoter and the coding region.

As illustrated in FIG. 2, the Smart CAR vector also comprises a component encoding T2A, a self-cleaving 2A peptide. The average length of T2A is 18-22 amino acids. T2A can be applied in expressions of more than one gene in cells. It has a high cleavage efficiency between genes upstream and downstream of the T2A peptide.

Examination of the Quality of mSA-CAR-T Cells

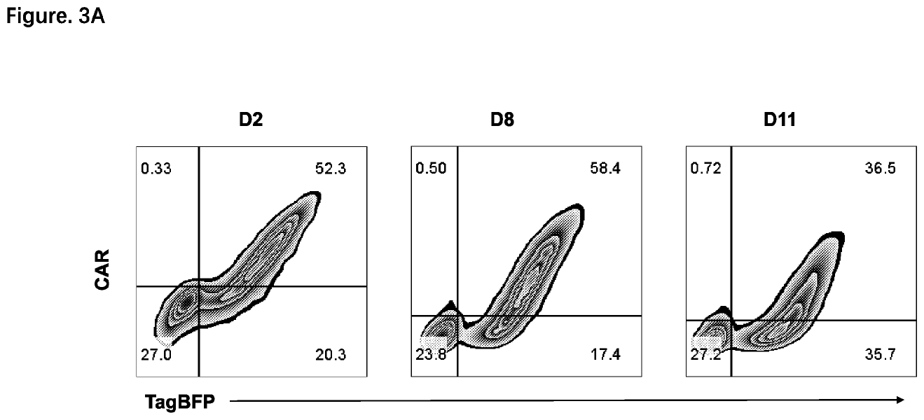

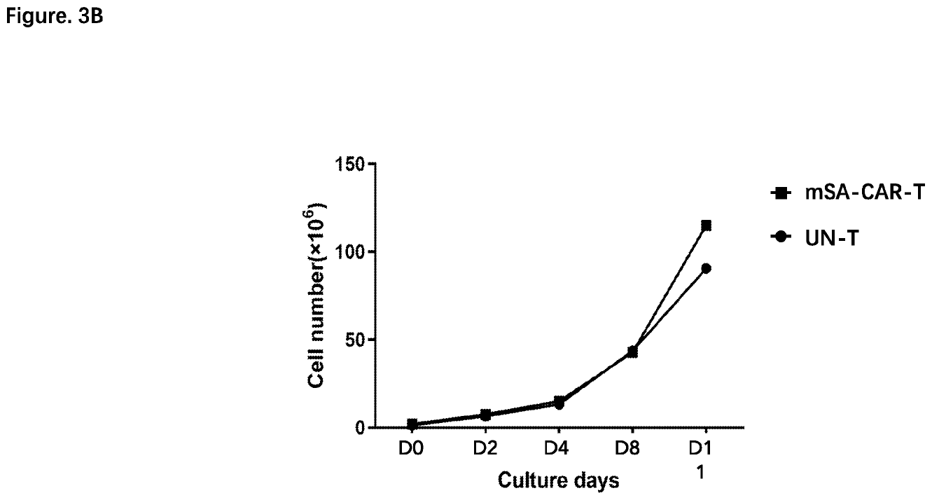

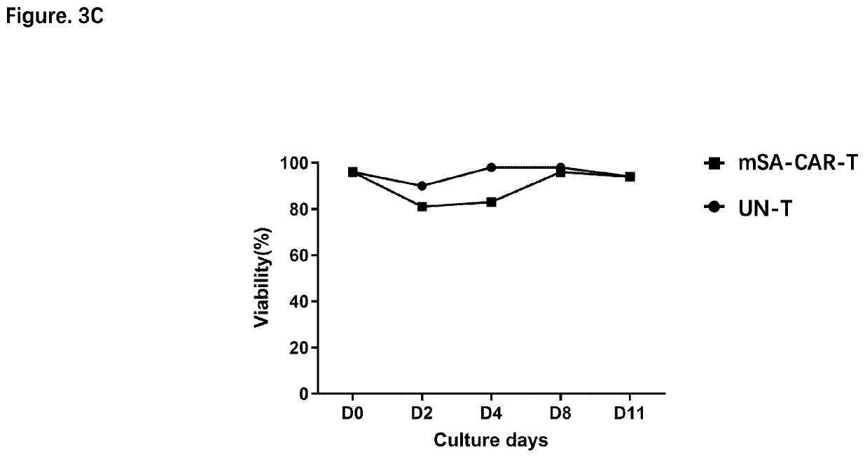

The CAR expression on T cells and cell proliferation were monitored. FITC-conjugated peptides with biotin modification at the N or C-terminus were used to detect the expression of the mSA-CAR on the surface of T cells (FIG. 3). Two FITC and biotin labeled peptides, Biotin-GGGGK(FITC)-NH₂ (SEQ ID NO: 8) and FITC-AhX-GGGGK(Biotin)-NH2, (SEQ ID NO: 9) were synthesized to quantify the mSA-CAR expression on T cell surface. No matter at which terminus of the peptide the biotin modification was located, the fluorescent biotin-labeled peptides were able to bind mSA-CAR-T with high efficiency.

After T cells were transduced by the mSA-CAR, Biotin-GGGGK(FITC)-NH2 (SEQ ID NO: 8) or FITC-AhX-GGGGK(Biotin)-NH2 (SEQ ID NO: 9) was added to the cell suspension and the cells were incubated. After a PBS wash, the transduction ratio and mSA-CAR expression were examined via flow cytometry.

As shown in FIG. 3A, the transduction efficiency of T cells by the mSA-CAR vector was indicated by the signal of tagBFP. The mSA-CAR expression on the surface of T cells was indicated by the FITC signal. As shown in FIG. 3A, the fluorescent signals remained stable on day 2, day 8, and day 11 after transduction, which indicated the stable and consistent mSA-CAR expression on the T cell surface.

We also found that the mSA-CAR expression did not affect T cell proliferation or viability (FIGS. 3B and 3C).

Labeling Efficiency of Exogenous Antigen Via mgSrtA on Tumor Cells In Vitro

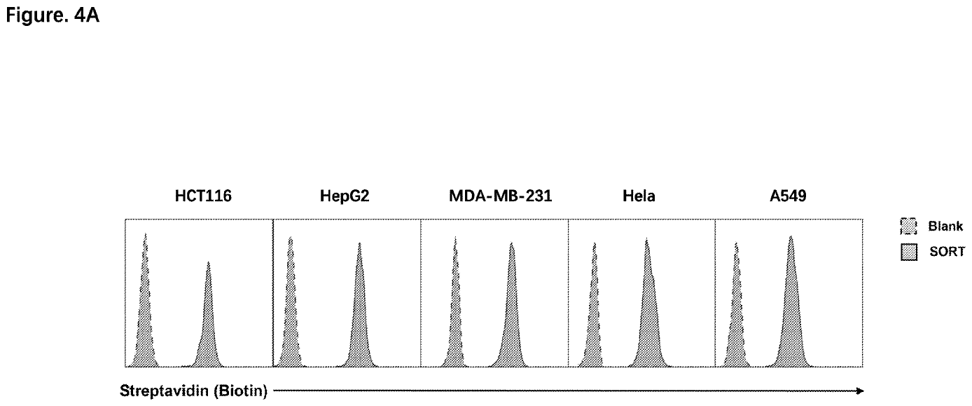

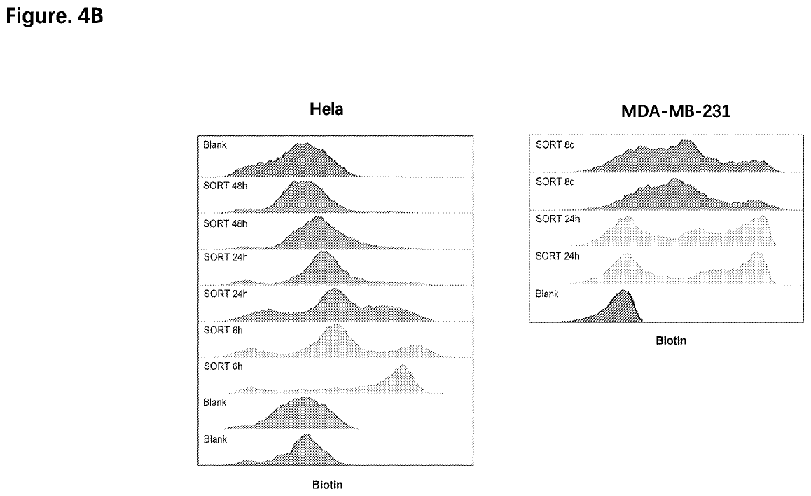

A biotin-modified exogenous antigen sortag was first mixed with mgSrtA in PBS buffer and then tumor cells were added into the PBS buffer containing the sortase and the exogenous antigen for sortagging. The tagging efficiency in tumor cells was tested after incubated with PE-streptavidin. Multiple tumor cell lines were shown to be efficiently labeled by the exogenous antigen within 30 mins in vitro, which was confirmed and quantified by flow cytometry (FIG. 4A).

Labeling Efficiency of Exogenous Antigen Via mgSrtA on Tumor Cells In Vivo

We established a cell line-derived xenograft (CDX) model to test in vivo labeling by exogenous antigens. For Hela tumor cell sortagging, one dose of intra-tumor injection (40 ul in volume, containing mgSrtA at 100 uM and Biotin-AALPETG*G (SEQ ID NO: 14) at 2 mM) was given; for MDA-MB-231 tumor cell sortagging, intra-tumor injection was given every 2 days for 3 times. At certain time points (as illustrated in FIG. 4B), the sortagged tumor was collected and cut into small fragments. After mashing through a 100 pm filter, a tumor cell suspension was collected and added with PE-streptavidin for Biotin-AALPETG*G (SEQ ID NO: 14) detection via flow cytometry.

After one dose of intratumor injection, the biotin signals of the successfully labeled antigen were detected on tumor cells after 6 hrs, and gradually decreased within 48 hrs (FIG. 4B, left). After three doses of intratumor injection were given, the biotin signals on tumor cells could last for more than one week (FIG. 4B, right).

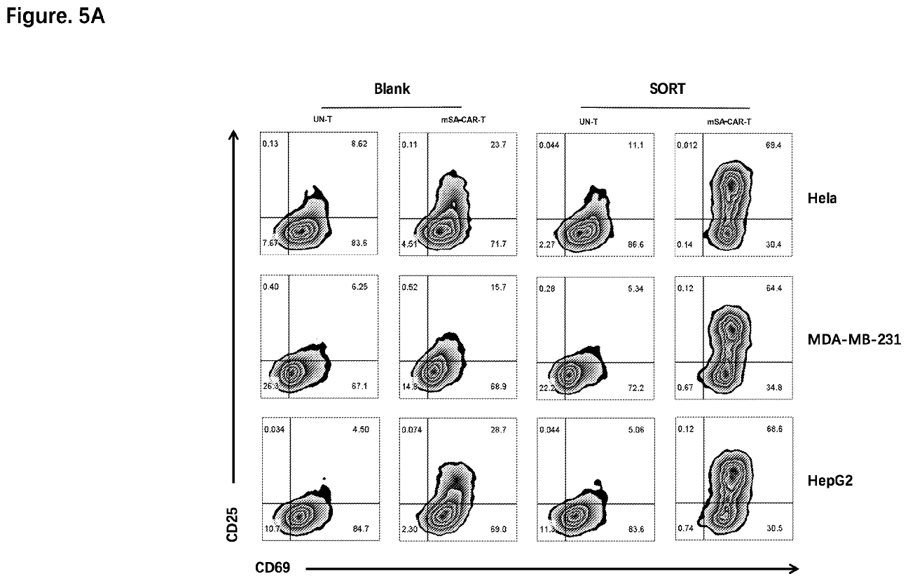

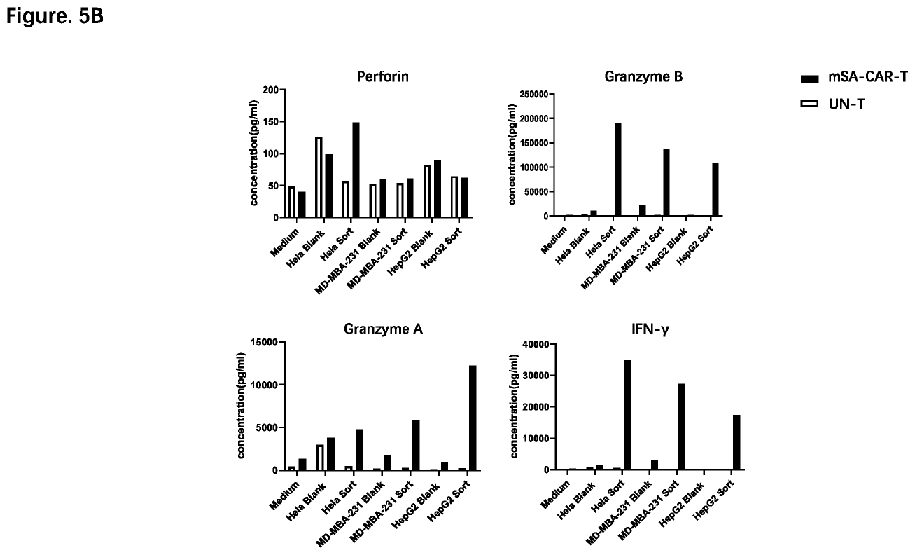

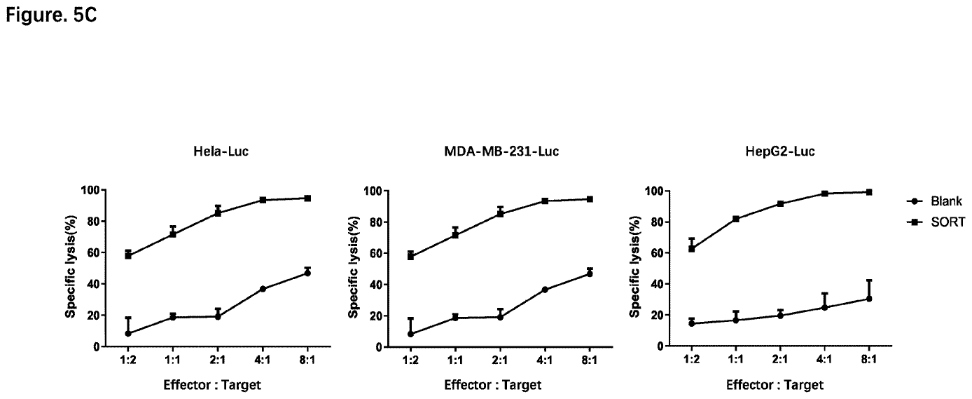

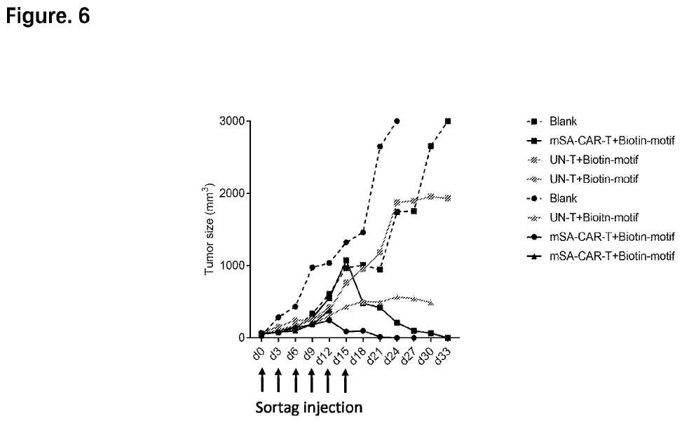

In the “activation phenotype assay,” the expression of the two active markers CD25 and CD69 on the T cell surface was tested by flow cytometry. After sortase-mediated tagging in vitro, 50,000 sortagged tumor cells were cocultured with mSA-CAR-T cells at an E:T ratio (E refers to effector cells such as engineered T cells; T refers to target cells such as tumor cells) as 2:1 in 200 ul RPMI-1640 per falcon tube. After 16-20 h of coculturing, the cell pellet was collected for the expression test of surface CD25 and CD69 and were tested by anti-CD25 and anti-CD69 antibody (Biolegend) via flow cytometry. The mSA-Smart-CAR T cells successfully recognized the exogenous antigens sortagged to the tumor cells. The mSA-CAR-T cells were subsequently activated, which resulted to an increased surface expression of CD25 and CD69 (FIG. 5A).

In the “cytokine release assay,” the concentrations of cytotoxic factors, including perforin, granzyme A, granzyme B, and IFN-γ, were tested in the supernatant of the tumor cells and mSA-CAR-T cell co-culture (FIG. 5B). After sortase-mediated tagging in vitro, 50,000 sortagged tumor cells were cocultured with mSA-CAR-T cells at an E:T ratio as 2:1 in 200 ul RPMI-1640 per falcon tube. After 16-20 h, the supernatant was collected for cytokine release assay (Legendplex human CD8/NK panel, Biolegend) via flow cytometry.

To perform the “cytotoxicity assay,” we engineered the target tumor cells to stably express luciferase. The luciferase was introduced to indicate the amount of survival tumor cells after being co-cultured with the mSA-CAR-T cells. After sortase-mediated tagging in vitro, 2000 sortagged tumor target cells were cocultured with mSA-CAR-T cells at different E:T ratio as 8:1, 4:1, 2:1, 1:1, and 1:2 in 50 ul PRMI-10% FBS per well of 384-well plate. After 16-20 h, the culture medium was removed to leave 25 ul per well, then 25 ul of a prepared luciferase detection reagent (L00877C, Genescript) was added to each well. Measurement was performed with a Varioskan LUX Microplate reader (Thermo).

The cytotoxicity of target tumor cells was calculated as follows: specific cytotoxicity=100×[(luminescence of remaining tumor cells after lysis by blank T cells−luminescence of remaining tumor cells after lysis by mSA-CAR-T cells)/(luminescence of remaining tumor cells after lysis by blank T cells)]; non-specific cytotoxicity=100×[(Total target cell luminescence−luminescence of remaining cells after lysis by blank T cells)/(Total target cell luminescence)]. Non-specific cytotoxicity should be controlled within 120%. Luciferin (the substrate of luciferase) was added after 16 hours of co-culture, and the biofluorescence was measured and converted to the percentage of specific cytotoxicity.

Apparently, the luciferase signal was negatively correlated with the cytotoxicity activities of the mSA-CAR-T cells. The three tumor cell lines examined in this assay all showed strong cytotoxicity after being co-cultured with the mSA-CAR-T cells, which indicated the effectiveness of the mSA-Smart CAR-T system (Figure SC).

### Example 2: PNEscFv-Smart CAR-T System

We demonstrated that tumor cells labeled with an exogenous antigen that was linked with a sortagging motif (e.g., -AALPETG*G (SEQ ID NO: 19)) were recognized and eliminated by the Smart CAR-T cells expressing the corresponding scFv that binds to the exogenous antigen.

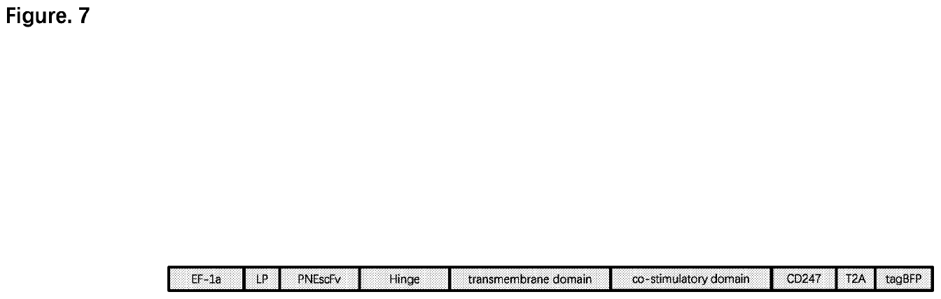

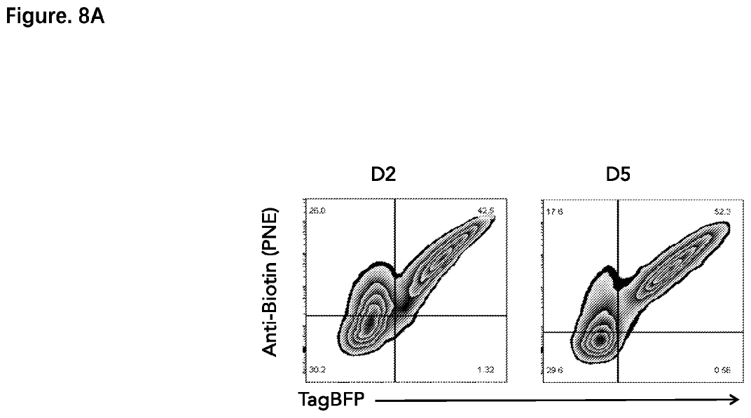

A 14-amino acid sequence (peptide neo-epitopes, PNE) from the yeast transcription factor GCN4 was used as the exogenous antigen in this Smart CAR-T system (PNE-CAR-T). PNE and the corresponding high-affinity scFv have been used in the past to establish switchable CAR-T cells (11). In the PNEscfv-Smart CAR-T system, as shown in the Smart CAR module illustrated in FIG. 7, the PNE scFv was merged with a hinge domain, transmembrane domain, co-stimulate domain, and CD247. A tagBFP (blue fluorescence protein) marker was included to mark the transduced T cells (FIG. 7). Similar to the demonstration of the mSA-CAR-T system, we conducted multiple experiments to illustrate the effectiveness of the PNEscFv-CAR-T system.

SEQ ID NO. 12 provides a vector encoding a PNEscfv-Smart CAR system:

(SEQ ID NO: 12)
GGCTCCGGTGCCCGTCAGTGGGCAGAGCGCACATCGCCCACAGTCCCCGAGAAGTT
GGGGGGAGGGGTCGGCAATTGAACCGGTGCCTAGAGAAGGTGGCGCGGGGTAAAC
TGGGAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATGGC
CCTTGCGTGCCTTGAATTACTTCCACCTGGCTGCAGTACGTGATTCTTGATCCCGAGC
TTCGGGTTGGAAGTGGGTGGGAGAGTTCGAGGCCTTGCGCTTAAGGAGCCCCTTCGC
CTCGTGCTTGAGTTGAGGCCTGGCCTGGGCGCTGGGGCCGCCGCGTGCGAATCTGGT
GGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGA
TCTGCACACTGGTATTTCGGTTTTTGGGGCCGCGGGCGGCGACGGGGCCCGTGCGTC
CCAGCGCACATGTTCGGCGAGGCGGGGCCTGCGAGCGCGGCCACCGAGAATCGGAC
GGGGGTAGTCTCAAGCTGGCCGGCCTGCTCTGGTGCCTGGCCTCGCGCCGCCGTGTA
TCGCCCCGCCCTGGGCGGCAAGGCTGGCCCGGTCGGCACCAGTTGCGTGAGCGGAA
AGATGGCCGCTTCCCGGCCCTGCTGCAGGGAGCTCAAAATGGAGGACGCGGCGCTC
GGGAGAGCGGGCGGGTGAGTCACCCACACAAAGGAAAAGGGCCTTTCCGTCCTCAG
CCGTCGCTTCATGTGACTCCACGGAGTACCGGGCGCCGTCCAGGCACCTCGATTAGT
TCTCGAGCTTTTGGAGTACGTCGTCTTTAGGTTGGGGGGAGGGGTTTTATGCGATGG
AGTTTCCCCACACTGAGTGGGTGGAGACTGAAGTTAGGCCAGCTTGGCACTTGATGT
AATTCTCCTTGGAATTTGCCCTTTTTGAGTTTGGATCTTGGTTCATTCTCAAGCCTCA
GACAGTGGTTCAAAGTTTTTTTCTTCCATTTCAGGTGTCGTGATCTAGAGCGCTGCCA
CCATGGCGCTTCCGGTCACTGCTTTGCTCCTGCCCCTGGCACTGCTGCTGCACGCCGC
TCGACCCGACGCCGTGGTGACCCAGGAGTCCGCCCTCACGTCATCCCCAGGGGAGA
CCGTCACCTTGACCTGCCGCTCTTCTACCGGCGCGGTTACCACAAGCAACTACGCGA
GTTGGGTGCAGGAGAAGCCCGACCACTTGTTCACCGGCCTCATCGGAGGAACTAAC
AACCGCGCTCCCGGGGTGCCTGCCCGCTTCTCTGGCTCCCTTATTGGGGACAAGGCC
GCCCTGACCATTACCGGTGCGCAGACTGAGGACGAGGCCATCTATTTCTGCGTGCTT
TGGTACAGTGACCACTGGGTCTTCGGCGGAGGGACCAAGCTGACCGTGCTGGGTGG
AGGCGGAGGCTCCGGTGGCGGGGGCAGCGGCGGTGGCGGCTCCGGAGGCGGCGGC
AGCGATGTACAGCTGCAGGAATCTGGACCCGGCCTGGTGGCTCCCTCTCAGAGTTTA
TCCATCACCTGCACTGTTTCCGGCTTCCTGCTGACTGATTACGGCGTCAACTGGGTGC
GCCAGAGCCCTGGGAAAGGTCTGGAGTGGCTAGGCGTGATTTGGGGCGATGGCATC
ACCGACTACAATTCGGCGCTCAAGTCGCGCCTGTCCGTGACTAAGGACAACTCCAA
GTCGCAGGTGTTCCTGAAGATGAACAGCCTTCAATCGGGTGATTCTGCTCGCTACTA
TTGCGTGACAGGGCTGTTCGATTACTGGGGCCAGGGCACCACCCTGACCGTGTCCTC
GGAGTCCAAGTACGGCCCTCCCTGTCCCCCGTGCCCAATCTACATATGGGCACCGCT
GGCCGGCACCTGTGGTGTCCTGTTGCTATCTCTGGTGATCACACTGTACTGTAAGCG
CGGCCGCAAGAAGCTGCTGTATATCTTCAAGCAGCCCTTCATGAGACCGGTGCAGA
CGACTCAGGAGGAAGATGGGTGCTCTTGTAGGTTTCCTGAGGAGGAGGAGGGCGGG
TGCGAGCTGCGCGTAAAATTTTCACGCAGCGCGGACGCACCCGCCTACAAGCAAGG
ACAGAACCAGCTCTACAACGAGCTCAACCTGGGCCGGCGTGAAGAGTACGATGTGC
TGGACAAGCGTCGCGGCCGCGACCCGGAGATGGGCGGCAAGCCGCGGCGTAAGAA
TCCACAGGAGGGGCTTTACAACGAGTTGCAGAAGGACAAAATGGCCGAGGCCTACT
CCGAGATCGGTATGAAGGGCGAGCGCCGTCGTGGTAAAGGGCATGACGGCCTTTAT
CAGGGGCTGTCCACGGCCACGAAAGACACCTACGACGCGCTGCATATGCAGGCCCT
TCCTCCTCGATGTACAGGCAGTGGAGAGGGCAGGGGAAGTCTGCTAACATGCGGGG
ACGTGGAGGAAAATCCCGGCCCCGGCAGCGAGCTGATTAAGGAGAACATGCACATG
AAGCTGTACATGGAGGGCACCGTGGACAACCATCACTTCAAGTGCACATCCGAGGG
CGAAGGCAAGCCCTACGAGGGCACCCAGACCATGAGAATCAAGGTGGTCGAGGGC
GGCCCTCTCCCCTTCGCCTTCGACATCCTGGCTACTAGCTTCCTCTACGGCAGCAAG
ACCTTCATCAACCACACCCAGGGCATCCCCGACTTCTTCAAGCAGTCCTTCCCTGAG
GGCTTCACATGGGAGAGAGTCACCACATACGAGGACGGGGGCGTGCTGACCGCTAC
CCAGGACACCAGCCTCCAGGACGGCTGCCTCATCTACAACGTCAAGATCAGAGGGG
TGAACTTCACATCCAACGGCCCTGTGATGCAGAAGAAAACACTCGGCTGGGAGGCC
TTCACCGAGACCCTGTACCCCGCTGACGGCGGCCTGGAAGGCAGAAACGACATGGC
CCTGAAGCTCGTGGGCGGGAGCCATCTGATCGCAAACATCAAGACCACATATAGAT
CCAAGAAACCCGCTAAGAACCTCAAGATGCCTGGCGTCTACTATGTGGACTACAGA
CTGGAAAGAATCAAGGAGGCCAACAACGAGACCTACGTCGAGCAGCACGAGGTGG
CAGTGGCCAGATACTGCGACCTCCCTAGCAAACTGGGGCACAAGCTTAATTAA

As illustrated in FIG. 7 and shown in the sequence of SEQ ID NO: 12, the nucleotides 1-1179 in SEQ ID NO: 12 represents EF-1a, the nucleotides 1197-1259 encodes LP (leading peptide), the nucleotides 1260-1976 encodes PNEscFv, the nucleotides 1989-2024 encodes Hinge, the nucleotides 2025-2096 encodes Transmembrane domain, the nucleotides 2097-2222 encodes co-stimulatory domain, the nucleotides 2223-2558 encodes CD247, the nucleotides 2574-2627 encodes T2A, the nucleotides 2628-3326 encodes tagBFP. Alternative nucleotide codons can be used to generate the PNEscfv-Smart CAR system.

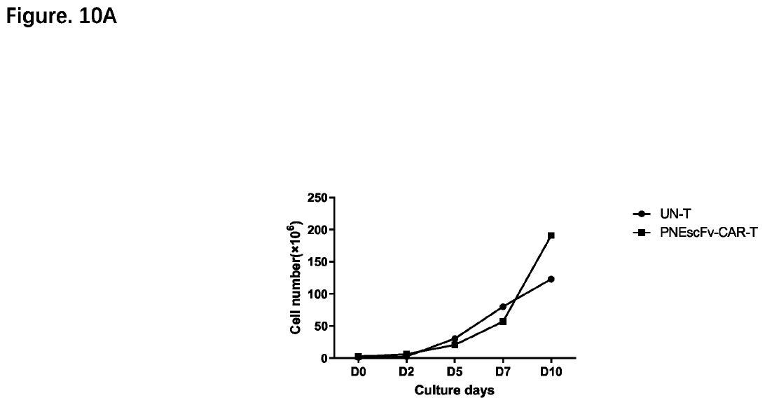

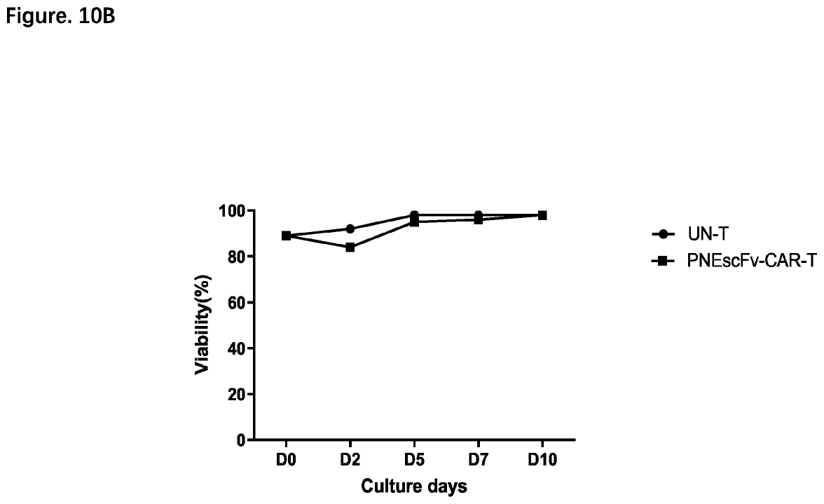

We also found that the PNE-CAR-T expression did not affect T cell proliferation (FIG. 10A) or viability (FIG. 10B).

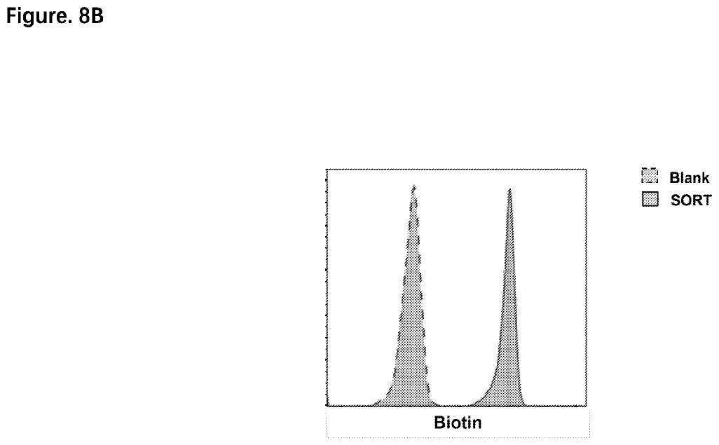

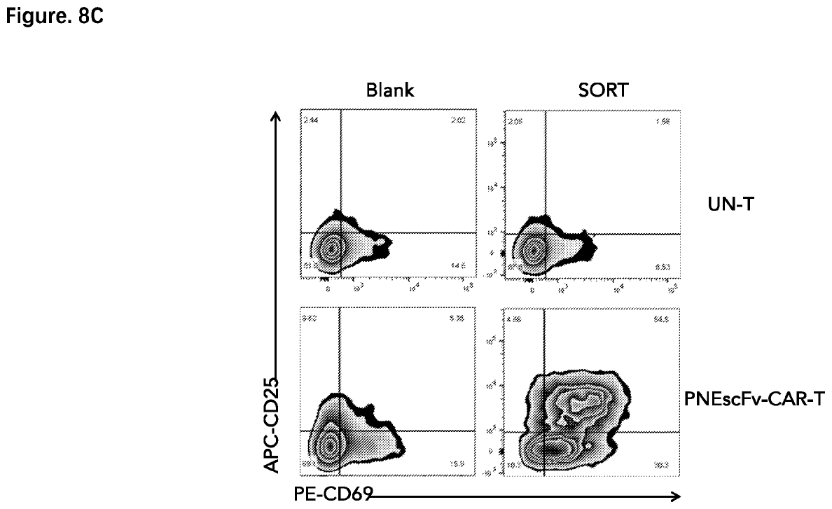

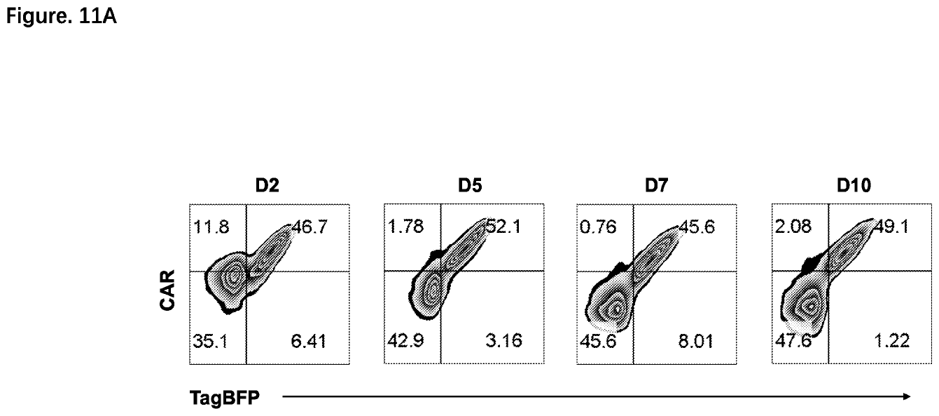

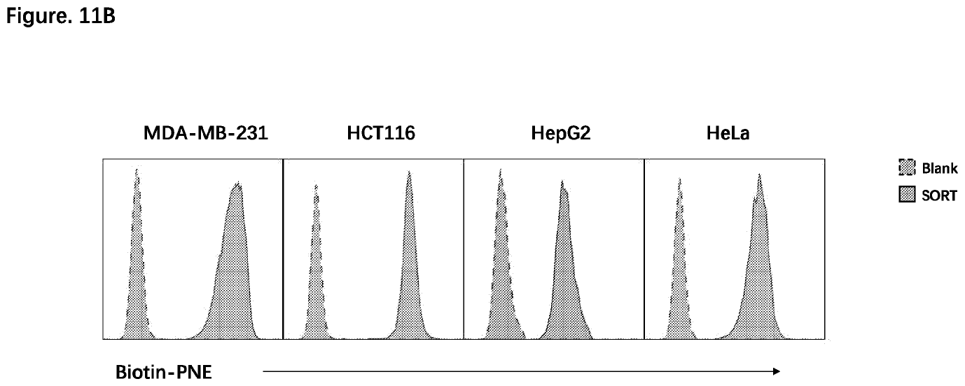

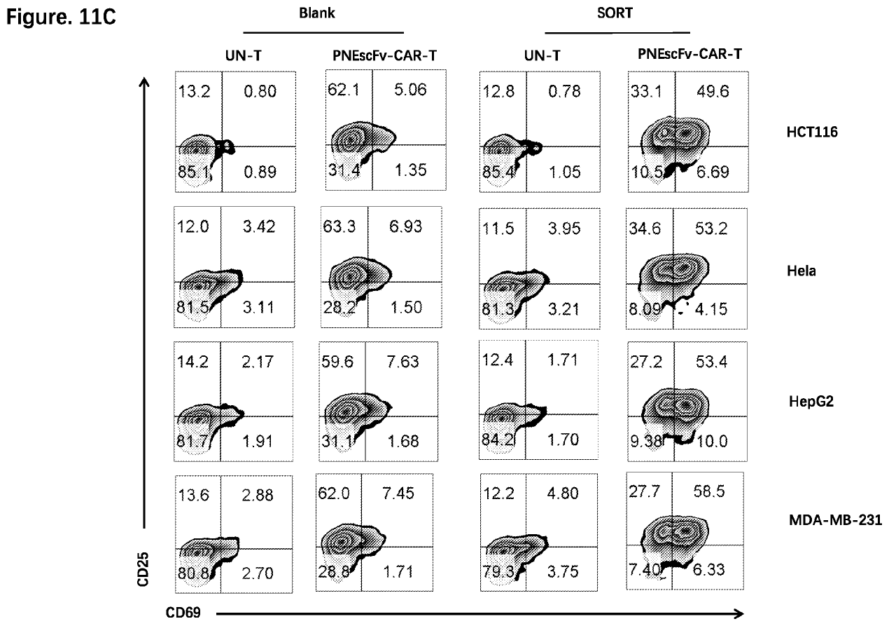

We examined the expression of PNEscFv-CAR on the T cell surface. Biotin-PNE-AALPETG*G followed by anti-biotin-PE was used for detection of the PNE scFv-CAR expression. After T cells were transduced with the PNEscFv-Smart CAR, Biotin-PNE-AALPETG*G was added in a T cell suspension and the cell suspension was incubated, and then anti-biotin-PE was added after a PBS wash. Transduction ratio and PNEscFv-CAR expression were tested via flow cytometry after a PBS wash. As shown in FIGS. 8A and 11A, the PNEscFv-CAR was stably expressed on the T cell surface.

We successfully labeled the PNE exogenous antigen to tumor cells in vitro (FIGS. 8B and 11B). Tumor cells were added into PBS buffer containing a sortase and Biotin-PNE-AALPETG*G (exogenous antigen) for sortagging. Then, the tagging efficiency in tumor cells was tested after incubation with anti-biotin-PE.

Next, we investigated the cytotoxicity induced by PNEscFv-Smart CAR-T cells against the sortagged tumor cells. After sortase-mediated tagging in vitro, 500,000 sortagged tumor target cells were cocultured with PNEscFv-CAR-T cells at an E:T ratio as 2:1 in 200 ul RPMI-1640 per falcon tube. After 16-20 h, the cell pellet was collected for the expression test of surface CD25 and CD69 (Biolegend) via flow cytometry. The cytotoxicity induced by PNEscFv-Smart CAR-T cells were determined using assays similar to those described in Example 1. For example, in the “activation phenotype assay,” the expression of two active markers CD25 and CD69 on the T cell surface was tested by flow cytometry. After PNE-AALPETG*G (SEQ ID NO: 16) labeling, the PNE on tumor cells specifically activated the PNEscFv-CAR T cells through antigen recognition, which was indicated by the increased surface expression of CD25 and CD69 (FIGS. 8C and 11C).

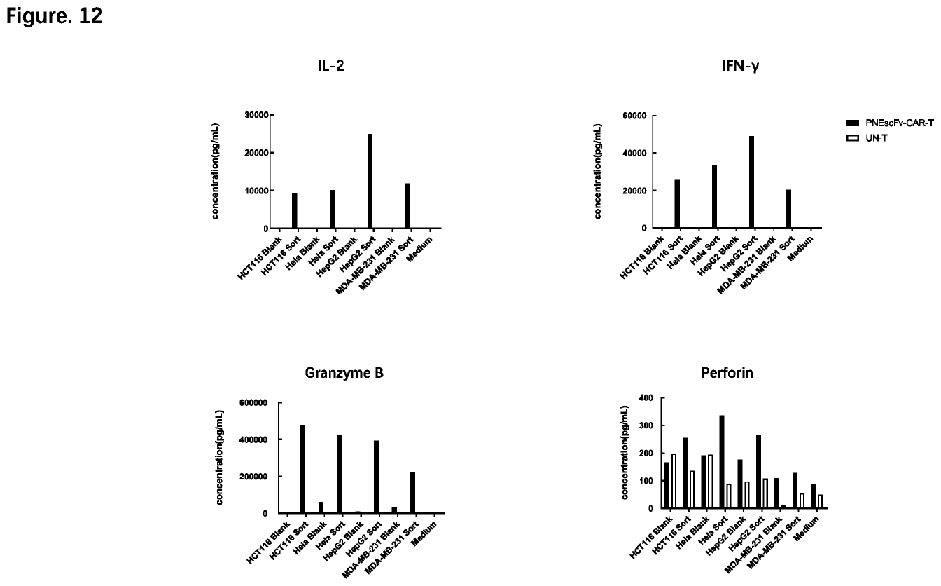

In a cytokine release assay. the concentrations of cytotoxic factors, including perforin, IL-2, granzyme B, and IFN-γ, were tested in the supernatant of the tumor cells and PNEscFv-CAR-T cell co-culture (FIG. 12). After sortase-mediated tagging in vitro, 50,000 sortagged tumor cells were cocultured with PNEscFv-CAR-T cells at an E:T ratio as 2:1 in 200 ul RPMI-1640 per falcon tube. After 16-20 h, the supernatant was collected for a cytokine release assay (Legendplex human CD8/NK panel, Biolegend) via flow cytometry.

To perform the cytotoxicity assay, we engineered the target tumor cells to stably express luciferase. The luciferase was introduced to indicate the amount of survival tumor cells after being co-cultured with the PNEscFv-CAR-T cells. After sortase-mediated tagging in vitro, 2000 sortagged tumor target cells were cocultured with PNEscFv-CAR-T cells at different E:T ratio as 8:1, 4:1, 2:1, 1:1, and 1:2 in 50 ul PRMI-10% FBS per well of a 384-well plate. After 16-20 h, the culture medium was removed to leave 25 ul per well, and then 25 ul of a prepared luciferase detection reagent (L00877C, Genescript) was added to each well. Measurement was performed with a Varioskan LUX Microplate reader (Thermo).

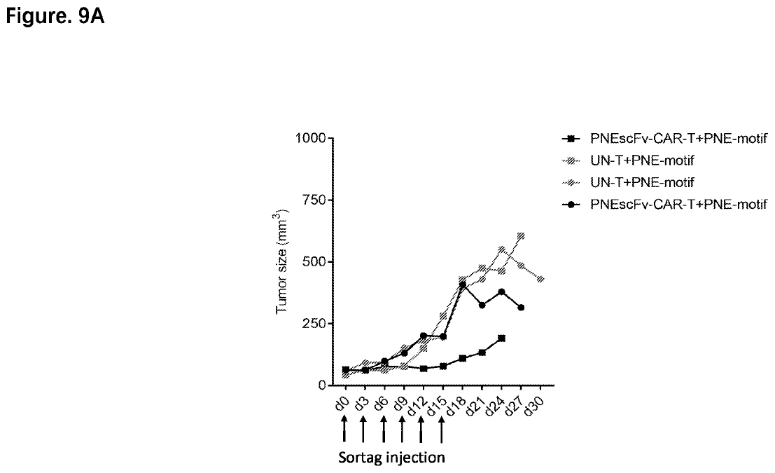

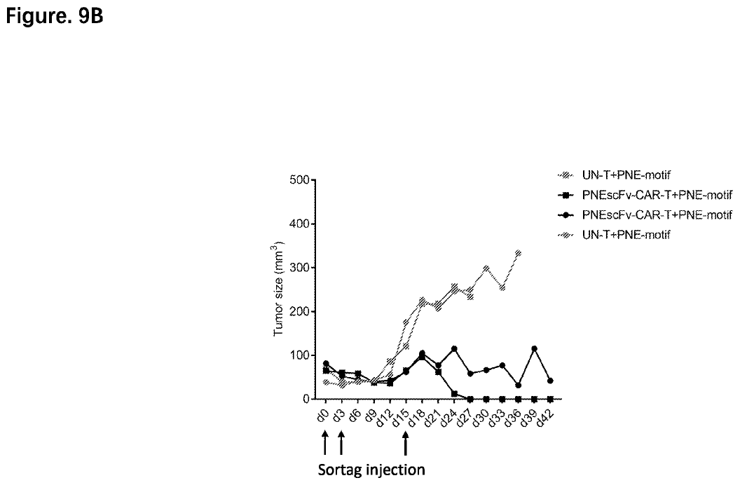

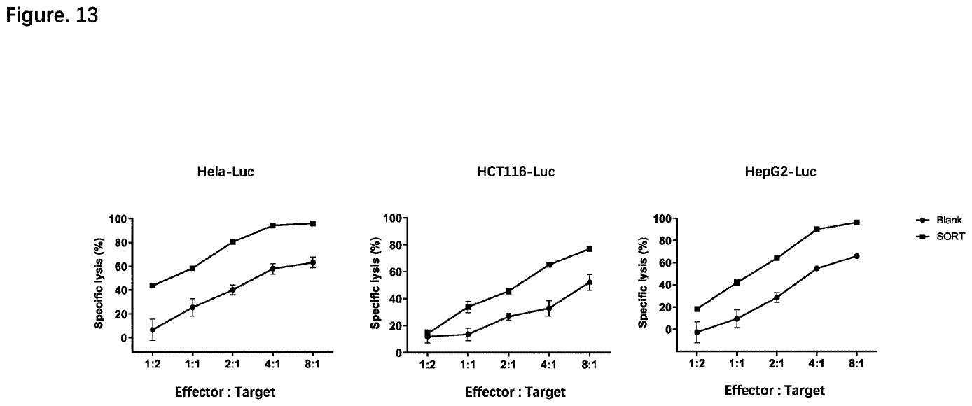

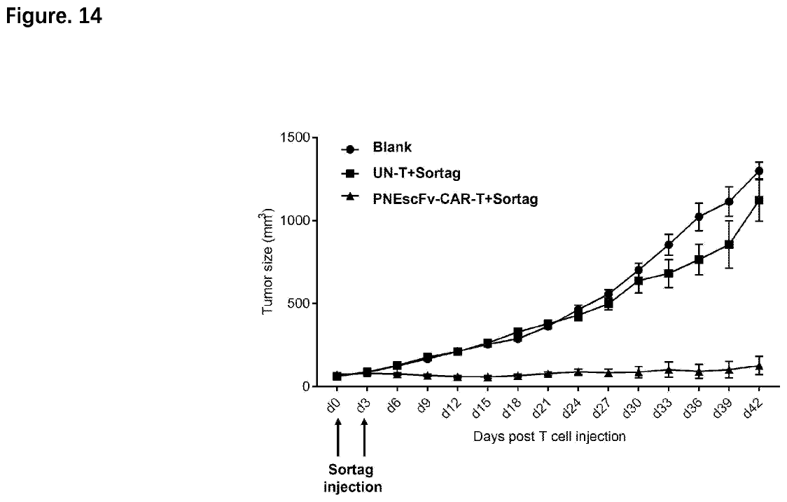

The cytotoxicity of target tumor cells was calculated as follows: specific cytotoxicity=100×[(luminescence of remaining tumor cells after lysis by blank T cells −luminescence of remaining tumor cells after lysis by PNEscFv-CAR-T cells)/(luminescence of remaining tumor cells after lysis by blank T cells)]; non-specific cytotoxicity=100×[(Total target cell luminescence−luminescence of remaining cells after lysis by blank T cells)/(Total target cell luminescence)]. Non-specific cytotoxicity should be controlled within ±20%. Luciferin (the substrate of luciferase) was added after 16 hours of co-culture, and the biofluorescence was measured and converted to the percentage of specific cytotoxicity. Apparently, the luciferase signal was negatively correlated with the cytotoxicity activities of the PNEscFv-CAR-T cells. The three tumor cell lines examined in this assay all showed strong cytotoxicity after being co-cultured with the PNEscFv-CAR-T cells, which indicated the effectiveness of the PNEscFv-CAR-T system (FIG. 13). In an animal model, we found the treatment with PNEscFv-CAR-T as well as sortag mixture intratumor injection could significantly control tumor size compared with UN-T treatment (FIGS. 9A, 9B, and 14).

### Example 3: Bi-CAR-T System

We demonstrated that tumor cells with low antigen expression labeled with an exogenous tag that was linked with a sortagging motif (e.g., -AALPETG*G) were recognized and eliminated by the Bi-CAR-T cells expressing PNE scFv that binds to the exogenous tag as well as an additional scFv that recognize tumor self-antigen (e.g. CLDN18.2 here).

In the Bi-CAR-T system, as shown in the Smart CAR module illustrated in FIG. 15, the PNE scFv as well as CLDN18.2 scFv were merged with a hinge domain, transmembrane domain, co-stimulate domain, and CD247. A tagBFP marker was included to mark the transduced T cells ( ).

SEQ ID NO: 13 provides a vector encoding a Bi-CAR system:

(SEQ ID NO: 13)
GGCTCCGGTGCCCGTCAGTGGGCAGAGCGCACATCGCCCACAGTCCCCGAGAAGTT
GGGGGGAGGGGTCGGCAATTGAACCGGTGCCTAGAGAAGGTGGCGCGGGGTAAAC
TGGGAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATGGC
CCTTGCGTGCCTTGAATTACTTCCACCTGGCTGCAGTACGTGATTCTTGATCCCGAGC
TTCGGGTTGGAAGTGGGTGGGAGAGTTCGAGGCCTTGCGCTTAAGGAGCCCCTTCGC
CTCGTGCTTGAGTTGAGGCCTGGCCTGGGCGCTGGGGCCGCCGCGTGCGAATCTGGT
GGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGA
TCTGCACACTGGTATTTCGGTTTTTGGGGCCGCGGGCGGCGACGGGGCCCGTGCGTC
CCAGCGCACATGTTCGGCGAGGCGGGGCCTGCGAGCGCGGCCACCGAGAATCGGAC
GGGGGTAGTCTCAAGCTGGCCGGCCTGCTCTGGTGCCTGGCCTCGCGCCGCCGTGTA
TCGCCCCGCCCTGGGCGGCAAGGCTGGCCCGGTCGGCACCAGTTGCGTGAGCGGAA
AGATGGCCGCTTCCCGGCCCTGCTGCAGGGAGCTCAAAATGGAGGACGCGGCGCTC
GGGAGAGCGGGCGGGTGAGTCACCCACACAAAGGAAAAGGGCCTTTCCGTCCTCAG
CCGTCGCTTCATGTGACTCCACGGAGTACCGGGCGCCGTCCAGGCACCTCGATTAGT
TCTCGAGCTTTTGGAGTACGTCGTCTTTAGGTTGGGGGGAGGGGTTTTATGCGATGG
AGTTTCCCCACACTGAGTGGGTGGAGACTGAAGTTAGGCCAGCTTGGCACTTGATGT
AATTCTCCTTGGAATTTGCCCTTTTTGAGTTTGGATCTTGGTTCATTCTCAAGCCTCA
GACAGTGGTTCAAAGTTTTTTTCTTCCATTTCAGGTGTCGTGATCTAGAGCGCTGCCA
CCATGGCGCTTCCGGTCACTGCTTTGCTCCTGCCCCTGGCACTGCTGCTGCACGCCGC
TCGACCCCAGGTGCAGCTGCAGCAGCCTGGCGCTGAACTGGTCCGACCTGGCGCCTC
CGTGAAGCTGTCCTGCAAGGCCTCCGGCTACACCTTCACCAGCTACTGGATCAACTG
GGTCAAGCAGCGGCCTGGCCAGGGCCTGGAATGGATCGGCAACATCTACCCCTCCG
ACTCCTACACCAACTACAACCAGAAGTTCAAGGACAAGGCCACCCTGACCGTGGAC
AAGTCCTCCTCCACCGCCTACATGCAGCTGTCCAGCCCCACCTCCGAGGACTCCGCC
GTGTACTACTGCACCCGGTCCTGGCGGGGCAACTCCTTCGACTATTGGGGCCAGGGC
ACCACCCTGACAGTGTCCTCTGGAGGCGGAGGATCTGACGCCGTGGTGACCCAGGA
GTCCGCCCTCACGTCATCCCCAGGGGAGACCGTCACCTTGACCTGCCGCTCTTCTAC
CGGCGCGGTTACCACAAGCAACTACGCGAGTTGGGTGCAGGAGAAGCCCGACCACT
TGTTCACCGGCCTCATCGGAGGAACTAACAACCGCGCTCCCGGGGTGCCTGCCCGCT
TCTCTGGCTCCCTTATTGGGGACAAGGCCGCCCTGACCATTACCGGTGCGCAGACTG
AGGACGAGGCCATCTATTTCTGCGTGCTTTGGTACAGTGACCACTGGGTCTTCGGCG
GAGGGACCAAGCTGACCGTGCTGGGTGGAGGCGGAGGCTCCGGTGGCGGGGGCAG
CGGCGGTGGCGGCTCCGGAGGCGGCGGCAGCGATGTACAGCTGCAGGAATCTGGAC
CCGGCCTGGTGGCTCCCTCTCAGAGTTTATCCATCACCTGCACTGTTTCCGGCTTCCT
GCTGACTGATTACGGCGTCAACTGGGTGCGCCAGAGCCCTGGGAAAGGTCTGGAGT
GGCTAGGCGTGATTTGGGGCGATGGCATCACCGACTACAATTCGGCGCTCAAGTCGC
GCCTGTCCGTGACTAAGGACAACTCCAAGTCGCAGGTGTTCCTGAAGATGAACAGC
CTTCAATCGGGTGATTCTGCTCGCTACTATTGCGTGACAGGGCTGTTCGATTACTGG
GGCCAGGGCACCACCCTGGGCGGAGGTGGTAGCGACATCGTGATGACCCAGTCCCC
CTCCAGCCTGACCGTGACCGCTGGCGAGAAAGTGACCATGAGCTGCAAGTCCTCCC
AGTCCCTGCTGAACTCCGGCAACCAGAAGAACTACCTGACCTGGTATCAGCAGAAG
CCCGGCCAGCCCCCCAAGCTGCTGATCTACTGGGCCTCCACCCGCGAGTCTGGCGTG
CCCGATAGATTCACCGGCTCCGGCAGCGGCACCGACTTTACCCTGACCATCTCCAGC
GTGCAGGCCGAGGACCTGGCCGTGTATTACTGTCAGAACGACTACTCCTACCCCTTC
ACCTTCGGCTCTGGCACCAAGCTGGAAATCAAGACCGTGTCCTCGGAGTCCAAGTAC
GGCCCTCCCTGTCCCCCGTGCCCAATCTACATATGGGCACCGCTGGCCGGCACCTGT
GGTGTCCTGTTGCTATCTCTGGTGATCACACTGTACTGTAAGCGCGGCCGCAAGAAG
CTGCTGTATATCTTCAAGCAGCCCTTCATGAGACCGGTGCAGACGACTCAGGAGGAA
GATGGGTGCTCTTGTAGGTTTCCTGAGGAGGAGGAGGGCGGGTGCGAGCTGCGCGT
AAAATTTTCACGCAGCGCGGACGCACCCGCCTACAAGCAAGGACAGAACCAGCTCT
ACAACGAGCTCAACCTGGGCCGGCGTGAAGAGTACGATGTGCTGGACAAGCGTCGC
GGCCGCGACCCGGAGATGGGCGGCAAGCCGCGGCGTAAGAATCCACAGGAGGGGC
TTTACAACGAGTTGCAGAAGGACAAAATGGCCGAGGCCTACTCCGAGATCGGTATG
AAGGGCGAGCGCCGTCGTGGTAAAGGGCATGACGGCCTTTATCAGGGGCTGTCCAC
GGCCACGAAAGACACCTACGACGCGCTGCATATGCAGGCCCTTCCTCCTCGATGTAC
AGGCAGTGGAGAGGGCAGGGGAAGTCTGCTAACATGCGGGGACGTGGAGGAAAAT
CCCGGCCCCGGCAGCGAGCTGATTAAGGAGAACATGCACATGAAGCTGTACATGGA
GGGCACCGTGGACAACCATCACTTCAAGTGCACATCCGAGGGCGAAGGCAAGCCCT
ACGAGGGCACCCAGACCATGAGAATCAAGGTGGTCGAGGGCGGCCCTCTCCCCTTC
GCCTTCGACATCCTGGCTACTAGCTTCCTCTACGGCAGCAAGACCTTCATCAACCAC
ACCCAGGGCATCCCCGACTTCTTCAAGCAGTCCTTCCCTGAGGGCTTCACATGGGAG
AGAGTCACCACATACGAGGACGGGGGCGTGCTGACCGCTACCCAGGACACCAGCCT
CCAGGACGGCTGCCTCATCTACAACGTCAAGATCAGAGGGGTGAACTTCACATCCA
ACGGCCCTGTGATGCAGAAGAAAACACTCGGCTGGGAGGCCTTCACCGAGACCCTG
TACCCCGCTGACGGCGGCCTGGAAGGCAGAAACGACATGGCCCTGAAGCTCGTGGG
CGGGAGCCATCTGATCGCAAACATCAAGACCACATATAGATCCAAGAAACCCGCTA
AGAACCTCAAGATGCCTGGCGTCTACTATGTGGACTACAGACTGGAAAGAATCAAG
GAGGCCAACAACGAGACCTACGTCGAGCAGCACGAGGTGGCAGTGGCCAGATACTG
CGACCTCCCTAGCAAACTGGGGCACAAGCTTAATTAA

As illustrated in FIG. 15B and shown in the sequence of SEQ ID NO: 13, the nucleotides 1-1179 in SEQ ID NO: 13 represents EF-la, the nucleotides 1197-1259 encodes LP (leading peptide), the nucleotides 1260-1613 encodes the heavy chain of CLDN18.2scFv, the nucleotides 1629-1955 encodes the light chain of PNEscFv, the nucleotide 2019-2345 encodes the heavy chain of PNEscFv, the nucleotide 2361-2699 encodes the light chain of CLDN18.2scFv, the nucleotides 2712-2747 encodes Hinge, the nucleotides 2748-2819 encodes Transmembrane domain, the nucleotides 2820-2945 encodes co-stimulatory domain, the nucleotides 2946-3281 encodes CD247, the nucleotides 3297-3350 encodes T2A, the nucleotides 3351-4049 encodes tagBFP. Alternative nucleotide codons can be used to generate the Bi-CAR system.

In an animal model carrying tumor with low antigen expression (10% of the tumor cells expressed CLDN18.2), we found the treatment with PNE/CLGN18.2-CAR-T (Bi-CAR-T) as well as sortag mixture intratumor injection could significantly control tumor size compared with UN-T treatment (FIG. 16).

### Example 4: Cell Line Preparation

A lentiviral vector carrying intracellular luciferase gene (lenti-EF-1α-luciferase-T2A-HygR) was generated. Then, various tumor cell lines were transduced with the luciferase-carrying lentivirus. After 48 h post transduction, the cells were selected by the addition of hygromycin in culture medium. Then, the luciferase-expressing cell lines were kept culturing in a culture medium with hygromycin at 2 ug/ml.

### Example 5: Primary T Cell Isolation and Culture

Human PBMCs from healthy donors were obtained by Shanghai Liquan Hospital and isolated by Milestone Biotechnologies, which were approved for research purposes. Human pan T cells, including CD4+ T cells and CD8+ T cells, were isolated from human PBMC via negative selection (130-096-535, Miltenyi). For long term culture, T cells were cultured in RPMI-10% FBS with 100 U/ml recombinant human IL-2 only. T cells were cryopreserved in CryoStor® CS10(07930, Stemcell Technologies).

### Example 6: Lentivirus Packaging and Transduction in T Cells

Lentivirus was packaged via transfecting plasmid, packaging plasmid psPAX2, and envelope plasmid pMD2.G. Viral supernatant was collected at 48 h and 72 h post transfection, passed through 0.454 m filter, and concentrated via ultracentrifuging at 70,000 g for 2 h at 4 °C. The concentrated supernatant was subsequently aliquoted and stored at −80 °C. Human T cells were thawed 2 days before transduction and cultured in RPMI-10% FBS (Gibco) with T cell activator (130-111-160, Miltenyi) and 100 U/ml recombinant human IL-2 (200-02, Peprotech). 48 h later, lentivirus transduction was performed in the presence of Lentiboost-P at 4 mg/ml during spinfection at 2,000 g for 2 h at RT. After spinfection, T cells were cultured at 37 °C overnight and transferred into G-REX for long term culture. At different timepoints after transduction, T cells were collected for transduction efficiency test and CAR expression test via flow cytometry.

### Example 7: Transduction Ratio and Smart-CAR Expression Test

The CAR expression on Smart-CAR T cells was monitored on different days post transduction. In the mSA-Smart CAR-T system, FITC-AhX-GGGGK(Biotin)-NH2 (SEQ ID NO: 9) were synthesized as detectors. The detector was added in the cell suspension at 1 uM and the cell suspension was incubated at 4 °C for 20 min. After washing by PBS, the transduction ratio and Smart-CAR expression on the T cell surface were tested via flow cytometry (Cytoflex S, Beckman). In PNE-Smart CAR-T system, a PNE peptide with biotin modification was synthesized as a detector. The detector was added in the cell suspension at 1 uM and incubated with the cells at 4 °C for 20 min. After washing by PBS, anti-biotin-PE (Biolegend) was added in the cell suspension and incubated with the cells at 4 °C for 20 min. Then, cells were washed by PBS again. The transduction ratio and Smart-CAR expression on cell surface were tested via flow cytometry.

### Example 8: Sortase Production

Sortase (mgSrtA) was expressed and purified from transformed E coli BL21(DE3). A DNA sequence encoding mgSrtA was cloned into a pET-28a(+) vector and expressed in E. coli BL21(DE3) cells. Transformed cells were induced with 1 mM IPTG until OD600 reached 0.6. Cells were grown for another 3-4 hours at 37 °C before harvested by centrifugation followed by resuspension in a lysis buffer (20 mM Tris-HCl, pH 7.8, and 500 mM NaCl). Lysate after sonication was loaded onto an Ni-NTA column (Histrap 5 ml, GE Healthcare), which was washed with 30 ml of a washing buffer (20 mM Tris-HCl, pH 7.8, 500 mM NaCl, and 40 mM imidazole) and then the mgSrtA protein was eluted with an elution buffer (20 mM Tris-HCl, pH 7.8, 500 mM NaCl, and 250 mM imidazole). Glycerol was added to the purified mgSrtA protein to a final concentration of 10%, and the mgSrtA samples were aliquoted and stored at-80 °C.

### Example 9: Cell Surface Sortagging In Vitro

A Sortag reaction was performed in PBS at a total volume of 50 ul at 37 °C for 30 min. Unless otherwise described, sortase was used at a final concentration of 20 uM and sortag substrate (exogenous antigen) at 200 uM, with cell density at 1×10⁷/ml. After sortagging, the cells were washed 3 times by PBS. For the tagging efficiency test, cells were incubated with anti-biotin-PE (Biolegend) or PE-streptavidin (Biolegend) on ice for 15 min, washed again, and then analyzed via flow cytometry.

### Example 10: Cell Sortagging In Vivo

In each injection dose, the mgSrtA and biotinized sortag were mixed in 40 uL PBS at 100 uM and 2 mM respectively. In Hela sortagging, 1 dose of intra-tumor injection was given; in MDA-MB-231 sortagging, intra-tumor injection was given every 2 days for 3 times. At a given appointed time, tumor was collected and cut into small fragments. After mashing through 100 m filter, a tumor cell suspension was collected and washed by PBS. Then, PE-streptavidin or anti-biotin-PE was added in the cell suspension and incubated with the cells at 4 °C for 20 min. Then, the cells were washed by PBS again and detected for biotin on the cell surface via flow cytometry.

### Example 11: Cytokine Release Assay and Activation Assay for Smart-CAR T Cells

As the workflow demonstrated in FIG. 17, after sortase-mediated tagging in vitro, 5×10⁵ sortagged tumor target cells were co-cultured with smart-CAR T cells at an E:T ratio as 2:1 in 200 ul RPMI-1640 per falcon tube. After 16-20 h, the supernatant was collected for cytokine release assay (Legendplex human CD8/NK panel, Biolegend), whereas the cell pellet was collected for the expression test of surface CD25 and CD69 via flow cytometry.

### Example 12: Cytotoxicity Assay for Smart-CAR T Cells

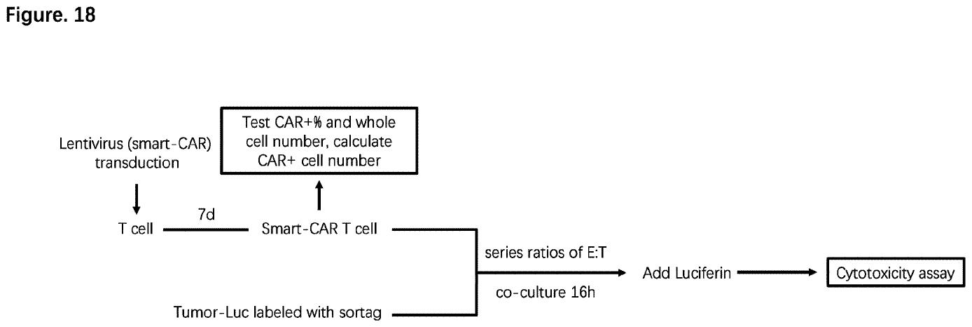

As the workflow demonstrated in FIG. 18, after sortase-mediated tagging in vitro, 2×10³ sortagged tumor target cells were co-cultured with smart-CAR T cells at an E:T ratio as 8:1 in 50 ul PRMI-10% FBS per well of 384-well plate. After 16-20 h, the culture medium was removed to leave 25 ul per well, and then 25 μl of a prepared luciferase reagent (L00877C, Genescript) was added to each well. Measurement was performed with a Varioskan LUX Microplate reader (Thermo). The cytotoxicity of target tumor cells was calculated as follows: specific cytotoxicity=100×[(luminescence of remaining cells after lysis by blank T cells−luminescence of remaining cells after lysis by Smart-CAR T cells)/(luminescence of remaining cells after lysis by blank T cells)]; non-specific cytotoxicity=100×[(Total target cell luminescence−luminescence of remaining cells after lysis by blank T cells)/(Total target cell luminescence)]. Non-specific cytotoxicity should be controlled within ±20%.

### Example 13: Xenograft Mouse Model

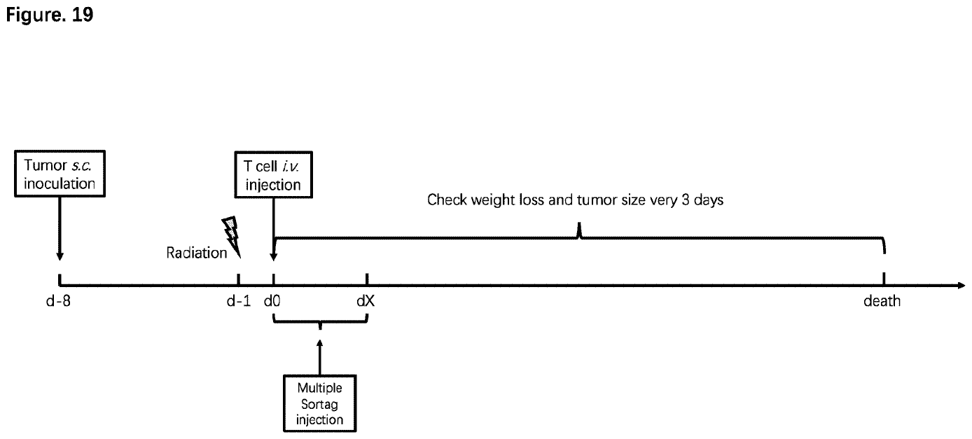

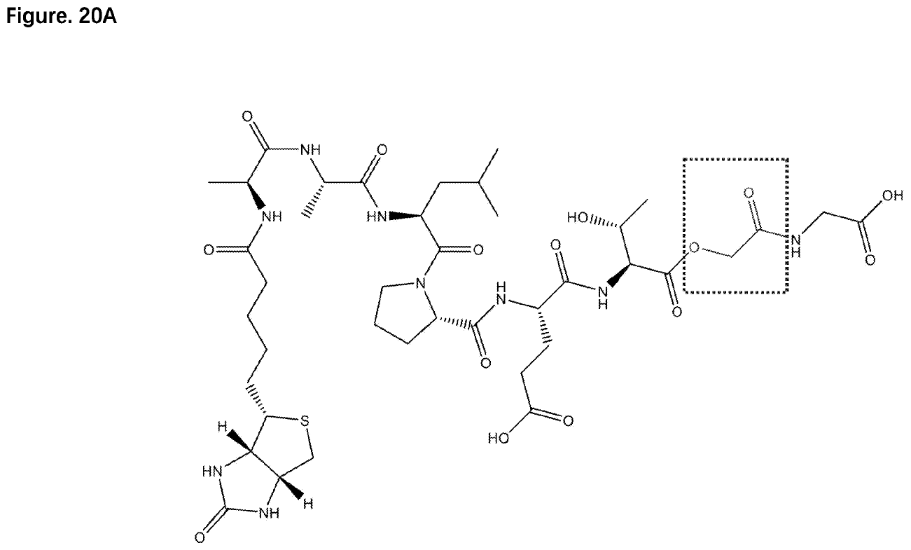

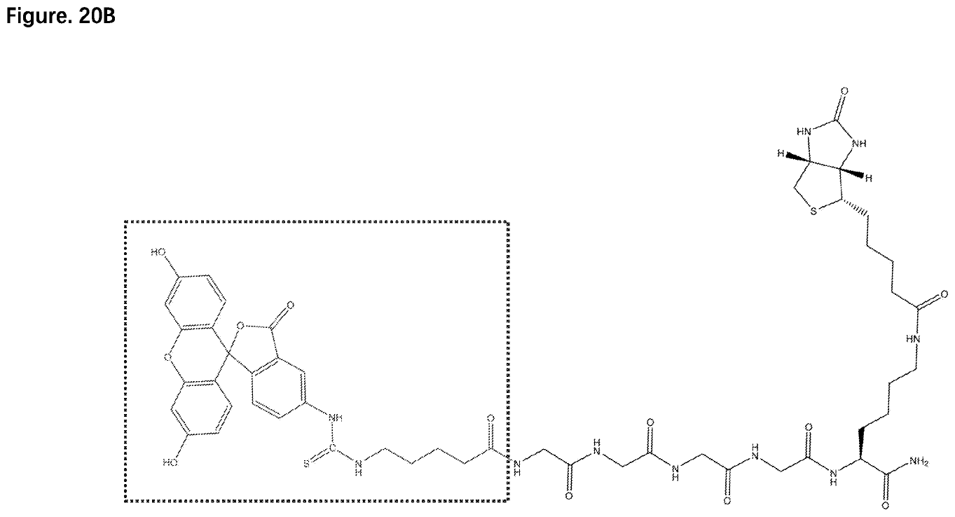

The animal experiments were performed as illustrated in the workflow in FIG. 19. NOD-Prkdc^{scid}Il2rg^{null}/Shjh (NPSG) mice were purchased from Shanghai Jihui Laboratory Animal Care Co., Ltd., and housed in standard individually ventilated and pathogen-free conditions in the laboratory animal resource center of Westlake University. Tumor-transplantation were conducted in the NOD-Prkdc^{scid}Il2rg^{null}/Shjh (NPSG) mice, female, between 6-8 weeks of age. All mice were used in accordance with the Institutional Animal Care and Use Committee (IACUC) guidelines for Westlake University. Tumor cells expressing luciferase were mixed with matrigel (1:1 volume) and injected subcutaneously into the right flank of the NPSG mice at 0.5×10⁶ or 1×10⁶ cells per recipient. After 7~10 days or after tumor burden reached 50 mm³, 3×10⁶ Smart CAR T cells (Including mSA-Car-T cells, PNEscFv-CAR-T cells or PNE/CLDN18.2-CAR-T cells) were adoptively transferred into each recipient via tail vein injection.

24 h later, an intra-tumor injection of mgSrtA and a Biotin-AALPETG*G mixture (SEQ ID NO: 14) (or mgSrtA and a PNE-AALPETG*G (SEQ ID NO: 16) mixture) was given to the mice every 3 days for multiple times. For each mouse, the mixture was prepared in 40 ul PBS per dose with mgSrtA at the final concentration as 10 uM and peptide substrate at the final concentration as 200M. Body weight loss and tumor size were measured every 3 days after T cell injection.

Further embodiments are illustrated below.
1. A method of treating a tumor in a subject in need thereof, comprising:
providing a sortase and an exogenous antigen to the tumor, wherein the exogenous antigen comprises an exogenous antigen ligand and a sortag motif, and wherein cells in the tumor are sortagged by the exogenous antigen, and
administering a therapeutically effective amount of genetically engineered immune cells that express a chimeric antigen receptor (CAR) comprising an antigen binding domain that is capable of binding the exogenous antigen.
2. The method of embodiment 1, wherein the CAR further comprises a binding domain that is capable of binding an endogenous antigen in the tumor.
3. The method of embodiment, wherein the endogenous antigen is claudin8.2.
4. The method of any one of embodiments 1-3, wherein the immune cells are T cells.
5. The method of any one of embodiments 1-4, wherein the immune cells were harvested from the subject before genetically engineered with the CAR.
6. The method of any one of embodiments 1-5, wherein the sortase is selected from sortase A, sortase B, and variants thereof.
7. The method of any one of embodiments 1-6, wherein the sortase is mgSrtA.
8. The method of any one of embodiments 1-7, wherein the exogenous antigen ligand is a peptide or a molecular label.
9. The method of any one of embodiments 1-8, wherein the sortag motif is selected from LPXTG (SEQ ID NO: 2), NP(Q/K)TN (SEQ ID NO: 3), (I/L)(P/A)XTG (e.g., LAXTG (SEQ ID NO: 7), IPXTG (SEQ ID NO: 17), IAXTG (SEQ ID NO: 18), LPXTG (SEQ ID NO: 2)), LPNTA (SEQ ID NO: 5), AALPETGXG (SEQ ID NO: 1), and LAXTG (SEQ ID NO: 7).
10. The method of any one of embodiments 1-9, wherein the sortase and the exogenous antigen are provided to the tumor or the vicinity of the tumor by injection.
11. The method of any one of embodiments 1-9, wherein the sortase and the exogenous antigen are provided to the tumor by intratumor injection.
12. The method of any one of embodiments 1-11, wherein the subject is a human.
13. The method of any one of embodiments 1-12, wherein the CAR comprises an antigen binding domain, a transmembrane domain, one or more costimulatory signaling region, and a cytoplasmic domain comprising an immunoreceptor tyrosine-based activation motif (ITAM), wherein the antigen binding domain is capable of binding the exogenous antigen.
14. The method of any one of embodiments 1-13, wherein the antigen binding domain is a ScFv that is capable of binding the exogenous antigen.
15. The method of any one of embodiments 1-14, wherein the sortase and the exogenous antigen are provided to the tumor sequentially.
16. The method of any one of embodiments 1-14, wherein a mixture of the sortase and the exogenous antigen are provided to the tumor.
17. A method of labeling a tumor cell with an exogenous antigen, comprising providing a sortase and an exogenous antigen to the tumor cell,
wherein the exogenous antigen comprises an exogenous antigen ligand and a sortag motif, and
wherein the tumor cell is sortagged by the exogenous antigen.
18. The method of embodiment 17, wherein the sortase is selected from sortase A, sortase B, and variants thereof.
19. The method of any one of embodiments 17-18, wherein the sortase is mgSrtA.
20. The method of any one of embodiments 17-19, wherein the exogenous antigen ligand is a peptide or a molecular label.
21. The method of any one of embodiments 17-20, wherein the sortag motif is selected from LPXTG (SEQ ID NO: 2), NP(Q/K)TN (SEQ ID NO: 3), (I/L)(P/A)XTG (e.g., LAXTG (SEQ ID NO: 7), IPXTG (SEQ ID NO: 17), IAXTG (SEQ ID NO: 18), LPXTG (SEQ ID NO: 2)), LPNTA (SEQ ID NO: 5), AALPETGXG (SEQ ID NO: 1), and LAXTG (SEQ ID NO: 7).
22. The method of any one of embodiments 17-21, wherein the tumor cell is selected from primary cells and immortalized cells.
23. The method of any one of embodiments 17-22, wherein the sortase and the exogenous antigen are injected to the tumor cell or the vicinity of the tumor cell.
24. The method of any one of embodiments 17-23, wherein the method of labeling occurs in vivo in a subject.
25. The method of any one of embodiments 17-23, wherein the method of labeling occurs in vitro.
26. The method of any one of embodiments 17-25. wherein the sortase and the exogenous antigen are provided to the tumor cell sequentially.
27. The method of any one of embodiments 17-25, wherein a mixture of the sortase and the exogenous antigen are provided to the tumor cell.
28. A tumor cell sortagged with an exogenous antigen by the method of any one of embodiments 17-27.
29. A tumor cell sortagged with an exogenous antigen.
30. The tumor cell of any one of embodiments 28-29, wherein the exogenous antigen is conjugated to the plasma membrane of the tumor cell.
31. An exogenous antigen comprising a sortag motif and an exogenous antigen ligand.
32. The exogenous antigen of embodiment 31, wherein the exogenous antigen ligand is a peptide or a molecular label.
33. A nucleic acid encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding the exogenous antigen of any one of embodiments 31-33.
34. The nucleic acid of embodiment 33, wherein the CAR further comprises a binding domain that is capable of binding an endogenous antigen in a tumor.
35. The nucleic acid of embodiment 34, wherein the endogenous antigen is claudin8.2.
36. The nucleic acid of any one of embodiments 33-35, wherein the cytoplasmic domain comprises a CD247 cytoplasmic domain.
37. The nucleic acid of any one of embodiments 34-36, wherein the one or more costimulatory signaling regions are selected from CD28, CD27, CD134 (OX40), and CD137 (4-1BB).
38. A vector comprising the nucleic acid of any one of embodiments 33-37.
39. A human immune cell comprising the nucleic acid of any one of embodiments 33-37 or the vector of embodiment 38.
40. The human immune cell of embodiment 39, wherein the human immune cell is a T cell from a human subject having tumor.
41. A pharmaceutical composition comprising a population of the human immune cell of embodiment 39 or 40.
42. A method of modifying an immune cell comprising delivering to the immune cell the vector of embodiment 38 or the nucleic acid of any one of embodiments 33-37.
43. A kit comprising a sortase and the exogenous antigen of any one of embodiments 31-32, wherein the kit optionally further comprises the nucleic acid of embodiments 33-37 or the vector of embodiment 38.
44. A genetically engineered immune cell for treating a tumor, wherein a cell in the tumor is sortagged by an exogenous antigen mediated by a sortase, wherein the exogenous antigen comprises an exogenous antigen ligand and a sortag motif, and wherein the genetically engineered immune cell expresses a chimeric antigen receptor (CAR) comprising an antigen binding domain that is capable of binding the exogenous antigen.
45. The genetically engineered immune cell for treating a tumor of claim 44, wherein the CAR further comprises a binding domain that is capable of binding an endogenous antigen in the tumor.
46. Use of a genetically engineered immune cell for the manufacture of a medicament for treating a tumor, wherein a cell in the tumor is sortagged by an exogenous antigen mediated by a sortase, wherein the exogenous antigen comprises an exogenous antigen ligand and a sortag motif, and wherein the genetically engineered immune cell expresses a chimeric antigen receptor (CAR) comprising an antigen binding domain that is capable of binding the exogenous antigen.
47. The use of a genetically engineered immune cell for the manufacture of a medicament for treating a tumor of claim 46, wherein the CAR further comprises a binding domain that is capable of binding an endogenous antigen in the tumor.

While the disclosure has been particularly shown and described with reference to specific embodiments, it should be understood by those having skill in the art that various changes in form and detail may be made therein without departing from the spirit and scope of the present disclosure as disclosed herein.

### REFERENCES

1. Martinez M, Moon E K. CAR T Cells for Solid Tumors: New Strategies for Finding, Infiltrating, and Surviving in the Tumor Microenvironment. Front Immunol. 2019; 10:128.
2. Marofi F, Motavalli R, Safonov V A, Thangavelu L, Yumashev A V, Alexander M, et al.
CAR T cells in solid tumors: challenges and opportunities. Stem Cell Res Ther. 2021; 12(1):81.
3. Sharma P, Hu-Lieskovan S, Wargo J A, Ribas A. Primary, Adaptive, and Acquired Resistance to Cancer Immunotherapy. Cell. 2017; 168(4):707-23.
4. Philip M, Schietinger A. CD8(+) T cell differentiation and dysfunction in cancer. Nat Rev Immunol. 2021.
5. Pishesha N, Ingram J R, Ploegh H L. Sortase A: A Model for Transpeptidation and Its Biological Applications. Annu Rev Cell Dev Biol. 2018; 34:163-88.
6. Zong Y, Bice T W, Ton-That H, Schneewind O, Narayana S V. Crystal structures of Staphylococcus aureus sortase A and its substrate complex. J Biol Chem. 2004; 279(30):31383-9.
7. Ge Y, Chen L, Liu S, Zhao J, Zhang H, Chen P R. Enzyme-Mediated Intercellular Proximity Labeling for Detecting Cell-Cell Interactions. J Am Chem Soc. 2019; 141(5):1833-7.
8. Marabelle A, Tselikas L, de Baere T, Houot R. Intratumoral immunotherapy: using the tumor as the remedy. Ann Oncol. 2017; 28(suppl_12):xii33-xii43.
9. Melero I, Castanon E, Alvarez M, Champiat S, Marabelle A. Intratumoural administration and tumour tissue targeting of cancer immnunotherapies. Nat Rev Clin Oncol. 2021; 18(9):558-76.
10. Champiat S. Tselikas L, Farhane S, Raoult T, Texier M, Lanoy E, et al. Intratumoral Immunotherapy: From Trial Design to Clinical Practice. Clin Cancer Res. 2021; 27(3):665-79.
11. Rodgers D T, Mazagova M, Hampton E N, Cao Y, Ramadoss N S, Hardy I R, et al. Switch-mediated activation and retargeting of CAR-T cells for B-cell malignancies. Proc Natl Acad Sci USA. 2016; 113(4):E459-68.
12. Bradshaw, W. J. et al. Molecular features of the sortase enzyme family. FEBS 1282, 2097-2114 (2015).
13. Chen, I., Dorr, B. M. & Liu, D. R. A general strategy for the evolution of bond-forming enzymes using yeast display. Proc Natl Acad Sci USA 108, 11399-11404 (2011).
14. Glasgow, J. E., Salit, M. L. & Cochran, J. R. In Vivo Site-Specific Protein Tagging with Diverse Amines Using an Engineered Sortase Variant. J Am Chem Soc 138, 7496-7499 (2016).
15. Chen, L. et al. Improved variants of SrtA for site-specific conjugation on antibodies and proteins with high efficiency. Sci Rep 6, 31899 (2016).
16. Ge, Y. et al. Enzyme-Mediated Intercellular Proximity Labeling for Detecting Cell-Cell Interactions. J Am Chem Soc 141, 1833-1837 (2019).
17. Podracky, C. J. et al. Laboratory evolution of a sortase enzyme that modifies amyloid-beta protein. Nat Chem Biol 17, 317-325 (2021).

1. A method of treating a tumor in a subject in need thereof, comprising:
providing a sortase and an exogenous antigen to the tumor, wherein the exogenous antigen comprises an exogenous antigen ligand and a sortag motif, and wherein cells in the tumor are sortagged by the exogenous antigen, and
administering a therapeutically effective amount of genetically engineered immune cells that express a chimeric antigen receptor (CAR) comprising an antigen binding domain that is capable of binding the exogenous antigen.

2. The method of claim 1, wherein the CAR further comprises a binding domain that is capable of binding an endogenous antigen in the tumor.

3. (canceled)

4. The method of claim 1, wherein the immune cells are T cells, NK cells, macrophages, dendritic cells, or B cells.

5. The method of claim 1, wherein the immune cells were harvested from the subject before genetically engineered with the CAR.

6. (canceled)

7. The method of claim 1, wherein the sortase is mgSrtA.

8. (canceled)

9. (canceled)

10. The method of claim 1, wherein the sortase and the exogenous antigen are provided to the tumor or the vicinity of the tumor by injection.

11. (canceled)

12. (canceled)

13. The method of claim 1, wherein the CAR comprises an antigen binding domain, a transmembrane domain, one or more costimulatory signaling region, and a cytoplasmic domain comprising an immunoreceptor tyrosine-based activation motif (ITAM), wherein the antigen binding domain is capable of binding the exogenous antigen.

14. The method of claim 1, wherein the antigen binding domain is an ScFv that is capable of binding the exogenous antigen.

15. The method of claim 1, wherein the sortase and the exogenous antigen are provided to the tumor sequentially or wherein a mixture of the sortase and the exogenous antigen are provided to the tumor cell.

16. (canceled)

17. A method of labeling a tumor cell with an exogenous antigen, comprising providing a sortase and an exogenous antigen to the tumor cell,
wherein the exogenous antigen comprises an exogenous antigen ligand and a sortag motif, and
wherein the tumor cell is sortagged by the exogenous antigen.

18. (canceled)

19. The method of claim 17, wherein the sortase is mgSrtA.

20. (canceled)

21. (canceled)

22. The method of claim 17, wherein the tumor cell is selected from primary cells and immortalized cells.

23. The method of claim 17, wherein the sortase and the exogenous antigen are injected to the tumor cell or the vicinity of the tumor cell.

24. The method of claim 17, wherein the method of labeling occurs in vivo in a subject or in vitro.

25. (canceled)

26. The method of claim 17, wherein the sortase and the exogenous antigen are provided to the tumor cell sequentially or wherein a mixture of the sortase and the exogenous antigen are provided to the tumor cell.

27. (canceled)

28. A tumor cell sortagged with an exogenous antigen by the method of claim 17.

29. A tumor cell sortagged with the exogenous antigen of claim 31.

30. The tumor cell of claim 28, wherein the exogenous antigen is conjugated to the plasma membrane of the tumor cell.

31. An exogenous antigen comprising a sortag motif and an exogenous antigen ligand.

32. (canceled)

33. A nucleic acid encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding the exogenous antigen of claim 31.

34. The nucleic acid of claim 33, wherein the CAR further comprises a binding domain that is capable of binding an endogenous antigen in a tumor.

35. (canceled)

36. (canceled)

37. (canceled)

38. A vector comprising the nucleic acid of claim 33.

39. A human immune cell comprising the nucleic acid of claim 33 or a vector comprising the nucleic acid of claim 33.

40. The human immune cell of claim 39, wherein the human immune cell is a T cell, NK cell, macrophage, dendritic cell, or B cell from a human subject having tumor.

41. A pharmaceutical composition comprising a population of the human immune cell of claim 39.

42. A method of modifying an immune cell comprising delivering to the immune cell the nucleic acid of claim 33 or a vector comprising the nucleic acid of claim 33.

43. A kit comprising a sortase and an exogenous antigen, wherein the exogenous antigen comprises a sortaq motif and an exogenous antigen ligand, and
wherein the kit optionally further comprises the nucleic acid of claim 33 or a vector comprising the nucleic acid of claim 33.

44-47. (canceled)


SEQUENCE LISTING (19 sequences)

SEQ ID NO: 1 - protein, 9 aa, synthetic construct
AALPETGXG

SEQ ID NO: 2 - protein, 5 aa, synthetic construct
LPXTG

SEQ ID NO: 3 - protein, 5 aa, synthetic construct
  VARIANT 3: note = X may be Q or K
NPXTN

SEQ ID NO: 4 - skipped

SEQ ID NO: 5 - protein, 5 aa, synthetic construct
LPNTA

SEQ ID NO: 6 - protein, 9 aa, synthetic construct
AALPETGXG

SEQ ID NO: 7 - protein, 5 aa, synthetic construct
LAXTG

SEQ ID NO: 8 - protein, 5 aa, synthetic construct
  MOD_RES 1: note = Labeled with biotin
  MOD_RES 5: note = Labeled with FITC
GGGGK

SEQ ID NO: 9 - protein, 5 aa, synthetic construct
  MOD_RES 1: note = labeled with FITC-AhX
  MOD_RES 5: note = labeled with biotin
GGGGK

SEQ ID NO: 10 - protein, 14 aa, synthetic construct
NYHLENEVARLKKL

SEQ ID NO: 11 - DNA, 2978 bp, synthetic construct
  source 1..2978: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagcgctgccaccatggagacagacacactcctgctatgggtgctgctgctctgggttccaggttccacaggtgacggcgccgaggctggcatcaccggtacctggtacaaccagcatggatcgacctttactgtcactgccggcgcggacggcaacctgaccggccagtatgagaaccgcgctcagggtactgggtgccagaacagcccttacaccctcactggccgctacaacggcaccaagctggagtggcgggtggagtggaacaactccacggagaattgtcacagccgcaccgagtggcgtggtcaataccagggcggggcagaggccaggattaatacccagtggaacttgacctacgagggcggctccgggccggccacagaacagggacaggatacgttcaccaaagtgaagccctctgcggcttccggatctaccgtgtcctcggagtccaagtacggccctccctgtcccccgtgcccaatctacatatgggcaccgctggccggcacctgtggtgtcctgttgctatctctggtgatcacactgtactgtaagcgcggccgcaagaagctgctgtatatcttcaagcagcccttcatgagaccggtgcagacgactcaggaggaagatgggtgctcttgtaggtttcctgaggaggaggagggcgggtgcgagctgcgcgtaaaattttcacgcagcgcggacgcacccgcctacaagcaaggacagaaccagctctacaacgagctcaacctgggccggcgtgaagagtacgatgtgctggacaagcgtcgcggccgcgacccggagatgggcggcaagccgcggcgtaagaatccacaggaggggctttacaacgagttgcagaaggacaaaatggccgaggcctactccgagatcggtatgaagggcgagcgccgtcgtggtaaagggcatgacggcctttatcaggggctgtccacggccacgaaagacacctacgacgcgctgcatatgcaggcccttcctcctcgatgtacaggcagtggagagggcaggggaagtctgctaacatgcggggacgtggaggaaaatcccggccccggcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaa

SEQ ID NO: 12 - DNA, 3329 bp, synthetic construct
  source 1..3329: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagcgctgccaccatggcgcttccggtcactgctttgctcctgcccctggcactgctgctgcacgccgctcgacccgacgccgtggtgacccaggagtccgccctcacgtcatccccaggggagaccgtcaccttgacctgccgctcttctaccggcgcggttaccacaagcaactacgcgagttgggtgcaggagaagcccgaccacttgttcaccggcctcatcggaggaactaacaaccgcgctcccggggtgcctgcccgcttctctggctcccttattggggacaaggccgccctgaccattaccggtgcgcagactgaggacgaggccatctatttctgcgtgctttggtacagtgaccactgggtcttcggcggagggaccaagctgaccgtgctgggtggaggcggaggctccggtggcgggggcagcggcggtggcggctccggaggcggcggcagcgatgtacagctgcaggaatctggacccggcctggtggctccctctcagagtttatccatcacctgcactgtttccggcttcctgctgactgattacggcgtcaactgggtgcgccagagccctgggaaaggtctggagtggctaggcgtgatttggggcgatggcatcaccgactacaattcggcgctcaagtcgcgcctgtccgtgactaaggacaactccaagtcgcaggtgttcctgaagatgaacagccttcaatcgggtgattctgctcgctactattgcgtgacagggctgttcgattactggggccagggcaccaccctgaccgtgtcctcggagtccaagtacggccctccctgtcccccgtgcccaatctacatatgggcaccgctggccggcacctgtggtgtcctgttgctatctctggtgatcacactgtactgtaagcgcggccgcaagaagctgctgtatatcttcaagcagcccttcatgagaccggtgcagacgactcaggaggaagatgggtgctcttgtaggtttcctgaggaggaggagggcgggtgcgagctgcgcgtaaaattttcacgcagcgcggacgcacccgcctacaagcaaggacagaaccagctctacaacgagctcaacctgggccggcgtgaagagtacgatgtgctggacaagcgtcgcggccgcgacccggagatgggcggcaagccgcggcgtaagaatccacaggaggggctttacaacgagttgcagaaggacaaaatggccgaggcctactccgagatcggtatgaagggcgagcgccgtcgtggtaaagggcatgacggcctttatcaggggctgtccacggccacgaaagacacctacgacgcgctgcatatgcaggcccttcctcctcgatgtacaggcagtggagagggcaggggaagtctgctaacatgcggggacgtggaggaaaatcccggccccggcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaa

SEQ ID NO: 13 - DNA, 4052 bp, synthetic construct
  source 1..4052: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagcgctgccaccatggcgcttccggtcactgctttgctcctgcccctggcactgctgctgcacgccgctcgaccccaggtgcagctgcagcagcctggcgctgaactggtccgacctggcgcctccgtgaagctgtcctgcaaggcctccggctacaccttcaccagctactggatcaactgggtcaagcagcggcctggccagggcctggaatggatcggcaacatctacccctccgactcctacaccaactacaaccagaagttcaaggacaaggccaccctgaccgtggacaagtcctcctccaccgcctacatgcagctgtccagccccacctccgaggactccgccgtgtactactgcacccggtcctggcggggcaactccttcgactattggggccagggcaccaccctgacagtgtcctctggaggcggaggatctgacgccgtggtgacccaggagtccgccctcacgtcatccccaggggagaccgtcaccttgacctgccgctcttctaccggcgcggttaccacaagcaactacgcgagttgggtgcaggagaagcccgaccacttgttcaccggcctcatcggaggaactaacaaccgcgctcccggggtgcctgcccgcttctctggctcccttattggggacaaggccgccctgaccattaccggtgcgcagactgaggacgaggccatctatttctgcgtgctttggtacagtgaccactgggtcttcggcggagggaccaagctgaccgtgctgggtggaggcggaggctccggtggcgggggcagcggcggtggcggctccggaggcggcggcagcgatgtacagctgcaggaatctggacccggcctggtggctccctctcagagtttatccatcacctgcactgtttccggcttcctgctgactgattacggcgtcaactgggtgcgccagagccctgggaaaggtctggagtggctaggcgtgatttggggcgatggcatcaccgactacaattcggcgctcaagtcgcgcctgtccgtgactaaggacaactccaagtcgcaggtgttcctgaagatgaacagccttcaatcgggtgattctgctcgctactattgcgtgacagggctgttcgattactggggccagggcaccaccctgggcggaggtggtagcgacatcgtgatgacccagtccccctccagcctgaccgtgaccgctggcgagaaagtgaccatgagctgcaagtcctcccagtccctgctgaactccggcaaccagaagaactacctgacctggtatcagcagaagcccggccagccccccaagctgctgatctactgggcctccacccgcgagtctggcgtgcccgatagattcaccggctccggcagcggcaccgactttaccctgaccatctccagcgtgcaggccgaggacctggccgtgtattactgtcagaacgactactcctaccccttcaccttcggctctggcaccaagctggaaatcaagaccgtgtcctcggagtccaagtacggccctccctgtcccccgtgcccaatctacatatgggcaccgctggccggcacctgtggtgtcctgttgctatctctggtgatcacactgtactgtaagcgcggccgcaagaagctgctgtatatcttcaagcagcccttcatgagaccggtgcagacgactcaggaggaagatgggtgctcttgtaggtttcctgaggaggaggagggcgggtgcgagctgcgcgtaaaattttcacgcagcgcggacgcacccgcctacaagcaaggacagaaccagctctacaacgagctcaacctgggccggcgtgaagagtacgatgtgctggacaagcgtcgcggccgcgacccggagatgggcggcaagccgcggcgtaagaatccacaggaggggctttacaacgagttgcagaaggacaaaatggccgaggcctactccgagatcggtatgaagggcgagcgccgtcgtggtaaagggcatgacggcctttatcaggggctgtccacggccacgaaagacacctacgacgcgctgcatatgcaggcccttcctcctcgatgtacaggcagtggagagggcaggggaagtctgctaacatgcggggacgtggaggaaaatcccggccccggcagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaa

SEQ ID NO: 14 - protein, 8 aa, synthetic construct
  MOD_RES 7: note = X is 2-hydroxyacetic acid
  MOD_RES 1: note = labeled with biotin
AALPETXG

SEQ ID NO: 15 - protein, 8 aa, synthetic construct
  MOD_RES 7: note = X is 2-hydroxyacetic acid
  MOD_RES 1: note = labeled by Biotin-PNE
AALPETXG

SEQ ID NO: 16 - protein, 8 aa, synthetic construct
  MOD_RES 1: note = labeled with PNE
  MOD_RES 7: note = X is 2-hydroxyacetic acid
AALPETXG

SEQ ID NO: 17 - protein, 5 aa, synthetic construct
IPXTG

SEQ ID NO: 18 - protein, 5 aa, synthetic construct
IAXTG

SEQ ID NO: 19 - protein, 8 aa, synthetic construct
  MOD_RES 7: note = X is 2-hydroxyacetic acid
AALPETXG